(19) United States
(12) Patent Application Publication
(10) Pub. No.: US 2004/0058356 A1
(43) Pub. Date: Mar. 25, 2004

(54) Methods for global profiling gene regulatory element activity

(76) Inventors: Mary E. Warren, Solana Beach, CA (US); Christopher Adams, San Diego, CA (US); Paul Labhart, San Diego, CA (US); Brian S. Egan, San Diego, CA (US); Marc Ballivet, Geneva (CH)

Correspondence Address: HALE AND DORR LLP, 300 PARK AVENUE, NEW YORK, NY, 10022, US

(21) Appl. No.: 10/426,734

(22) Filed: Apr. 30, 2003

Related U.S. Application Data
(63) Continuation-in-part of application No. PCT/IB02/00589, filed on Feb. 28, 2002.
(60) Provisional application No. 60/272,753, filed on Mar. 1, 2001.

(51) Int. Cl. C12Q 1/68

(52) U.S. Cl. 435/6

(57) ABSTRACT

The present invention provides methods for profiling, in a global manner, the activity of gene regulatory elements in cells, including eukaryotic and prokaryotic cells. The methods involve analysis of regulatory element complexes formed under cell-free conditions or within cells (sometimes called “in vivo”). In accordance with the invention, cells can be in any state of metabolism, resting, growing, normal, mutant, diseased or differentiating. The gene regulatory element activity profiles generated for the cells in different cell populations are compared to determine differences in gene regulatory activity and overall gene expression between or among different types or states of cells.

This application is a continuation-in-part of application Serial No. PCT/IB02/00589, filed Feb. 28, 2002, the contents of which are hereby incorporated by reference in their entirety. PCT/IB02/00589 claims the benefit of application Serial No. 60/272,753, filed Mar. 1, 2001.

### FIELD OF THE INVENTION

The present invention relates generally to monitoring gene regulation. More specifically, this invention relates to methods for determining, in a comprehensive manner, gene regulatory element activity in cells. Even more specifically, the invention relates to the global profiling of gene regulation in eukaryotic or prokaryotic cells from different sources, in various metabolic states of growth and/or differentiation, or after exposure to external changes, such as treatment with drugs or bioactive compounds, in order to identify differences in gene regulation between and among cells as a result of such metabolic states, exposure, and/or treatment.

### BACKGROUND OF THE INVENTION

In recent years, gene expression, and more specifically, the regulation of gene expression has generated intense interest in the art. Gene regulation is especially important because it is in involved in the fundamental control of cellular growth, differentiation and function, and organismic development. Similarly, aberrant gene regulation has been recognized as playing a leading role in the onset and progression of many disease states. Not only is it important to determine which genes are differentially expressed between and among biologically-relevant cells, it is useful to identify groups of coordinately regulated genes within the population of differentially expressed genes and to elucidate those mechanisms or factors that are responsible for the differential expression.

Understanding differential gene regulation and expression is important for determining differences or changes that occur between cells, including those involving mechanisms of disease, steps in development and differentiation, subtypes of diseases, changes that can be used for developing new therapies and/or diagnostic tests, events involved in disease progression and many other differences between cells, tissues and organisms. In addition, assays that can detect and quantify such differences are extremely useful for testing the effects of compounds on cells by measuring endpoints such as efficacy, toxicity, resistance, and mode of action.

A significant amount of gene regulation occurs at the level of gene transcription and, in particular, transcription initiation. Gene sequences include, or are adjacent to, promoter and enhancer sequences that bind transcriptional activator and repressor molecules that act to regulate the expression of the gene sequences associated therewith. Activator molecules, also called regulatory proteins, have been observed to bind to nucleic acid sequences, e.g., in the DNA, and to recruit molecular transcription initiation machinery to sites of transcription. In general, the initiation machinery includes RNA polymerase II and at least 50 other molecular components. Generally, the transcription initiation machinery includes proteins that bind DNA or other proteins, e.g., cyclin-dependent kinases that regulate polymerase activity, and acetylases and other enzymes that modify chromatin structure. Thus, it can be understood that gene expression is controlled by many selective protein-nucleic acid and protein-protein interactions.

Presently, gene regulation, as well as its mechanisms and consequences, are under investigation. However, there is a concurrent lack of knowledge regarding the complete set of transcriptional regulators and a lack of understanding concerning how these regulators interact and control the transcription machinery. Needed in the art is further knowledge concerning the various components involved in controlling the transcription machinery. Moreover, because cells must adjust their genetic expression to accommodate changes in environmental and metabolic conditions to provide for growth control, differentiation, and development, it is clear that methods are needed to assess the coordination of genomic expression, particularly, for example, in cells, tissues, and organs that have been stressed, diseased, exposed to various treatments or drugs, or mutated. Methods are also needed to determine which genes change in their expression as a result of internal or external influences, and particularly as a result of changes in the gene regulatory elements. Furthermore, such methods are needed to identify and quantify the above characteristics at a moderate to high throughput.

In the area of regulation of gene expression, it is now understood that a primary factor in the control of gene expression is the level of activity of trans-acting protein molecules (transcription factors that act as activators and repressors) that bind DNA in a sequence-specific manner. Further, other transcription factors or other regulatory proteins can specifically bind the DNA-binding factors to exert another level of control of gene expression. By assessing such protein-DNA and protein-protein binding activities in a cell at any given time, useful insight can be gained regarding changes in gene expression that occur as a result of changes in these types of protein binding activities. Insight can also be gained as to which genes are coordinately regulated under various conditions. However, until now, such activity has been assessed only for individual protein-DNA interactions, such as in yeast, using techniques such as electrophoretic mobility shift assay (EMSA) and footprinting.

For example, U.S. Pat. No. 6,410,233 B2, issued Jun. 25, 2002, to D. Mercola et al. discloses methods to identify nucleic acid molecules that correspond to genes that are regulated by a transcription factor. WO 01/16378 (Whitehead Institute for Biomedical Research) discloses a method of identifying one or more regions of a cellular genome that are bound by a protein. Neither U.S. Pat. No. 6,410,233 BS nor WO 01/16378 relates to a global profiling analysis of a wide variety of transcriptional regulatory elements in cells or compares the regulatory element activity of many regulatory complexes as they are found in cells, in different cell populations, or in cells in different states or conditions.

Given the still present and ongoing need in the art for methods to understand in a global fashion the regulatory mechanisms and elements involved in gene expression in cells, especially those regulatory elements that are related to diseases and responses to outside influences, the present invention provides methods for global analysis (i.e. profiling) of any given cell, cells, or population of cells.

### SUMMARY OF THE INVENTION

The present invention provides methods for determining the global profile of gene regulatory element activity in a cell population. This invention further includes the comparison of global profiles between or among cell populations to determine differences in gene regulation between or among those cell populations. In accordance with this invention, differences in gene regulation between and among the cell populations, as demonstrated by differential regulatory element activity, are used to identify and quantify the various intracellular activities that are related to both normal and diseased states and cellular responses to intracellular or extracellular signals. Such intracellular activities include differences in expression at the individual gene level, the co-regulation of sets of genes, the effects of internal changes and external influences, and pathological causes and effects involved in disease.

In one of its aspects, the present invention provides two avenues for forming specific regulatory element complexes that are analyzed to determine a regulatory element profile for any cell population. In one avenue, the complexes are formed outside of the cell in cell-free binding reactions to regenerate complexes that mimic those which are formed and found inside of cells. In the second avenue, the complexes are formed naturally inside of living cells and then are isolated, optionally, substantially purified, and analyzed. In either avenue, active gene regulatory elements and/or the gene sequences regulated by those elements can be identified by detecting and analyzing the specific regulatory complexes that are formed, resulting in a regulatory element profile for that cell type or population. These avenues also include methods for determining and identifying previously unknown regulatory elements, the activity of which comprises part or all of the regulatory element profile for that cell type or population.

The profile of regulatory element activity is informative regarding which elements are operating within any given cell or cell population, and to what level or extent of activity. One or more regulatory elements may be minimally controlling gene expression or having multitudes of effects within the cells. This can be considered the baseline of gene regulation and expression for that cell population, particularly, as to which genes are being controlled by which regulatory elements. Then, relevant comparisons are made between cells and cell populations, such as diseased cells versus nondiseased cells; cells at different stages of disease; cells exposed to external factors such as drug compounds versus cells not exposed; cells exposed to external factors for different amounts of time; and on and on.

By making these comparisons, it is possible to determine the gene regulatory activities that are different between the various types of cells, such as which regulatory elements are differential and which genes that they regulate are differentially expressed. In some cases, the regulatory element changes between cell populations might be significant and very meaningful, while in other cases, the changes may be less important; however, a global profiling scheme provides a way to understand and/or identify the significance, relevance, and import of changes in regulatory element activity and expression of the regulated genes, or the lack thereof. Global regulatory element profiling provides a way to understand the relationships of regulatory elements and expressed genes to each other. Also, global regulatory element profiles and RNA steady state levels, as determined by a variety of other methods referred to as RNA profiling, can complement and/or confirm each other. With the unknowns surrounding RNA profiling today, any method that can confirm differential RNA expression is valuable.

It is a particular aspect of the present invention to provide a cell-free global profiling method in which a protein extract containing nucleic acid binding factors and co-regulators from a cell population of interest is contacted with a plurality of nucleic acid molecules under conditions that allow the formation of specific cis site-regulatory protein complexes. Preferably, the plurality of nucleic acid molecules comprises more than two cis sites. For example, it can comprise a library of nucleic acids, containing at least two, and preferably different, cis sites.

In another aspect, the present invention provides methods that employ a library or libraries of nucleic acid molecules. The library(ies) can comprise a population of nucleic acid molecules containing known cis sites that bind nucleic acid binding factors. Alternatively, the library(ies) can comprise nucleic acid molecules that are not known to, but may contain, cis sites that bind nucleic acid binding factors. The methods include single- or double-stranded nucleic acid molecules, for example, RNA, DNA, and polynucleotide molecules that are found in genomic DNA or are representative of genomic DNA from a variety of eukaryotic and prokaryotic sources, nonlimiting examples of which include animals of all types, (e.g., mammals, vertebrates and invertebrates), plants, bacteria, archaebacteria, fungi, algae and viruses. According to the present invention, suitable nucleic acid molecules (i) can comprise molecules of a defined composition, for example, those including certain percentages of one or more nucleotides; (ii) can contain modified nucleotides, for example, methylated nucleotides, as well as, or alternatively, nucleotide analogs and derivatives; (iii) are synthetic or isolated from cells; (iv) can vary in length from about 4 to over about 1000 nucleotides or nucleotide pairs in length; (v) can comprise purified DNA or RNA, complementary DNA or cDNA, partially-purified DNA or RNA, or unpurified DNA or RNA; (vi) can comprise DNA within chromatin, a chromosome, or chromosome segment; and (vii) can comprise RNA in riboprotein complexes.

It is another particular aspect of the present invention to provide methods in which nucleic acids that are bound by transcription factor(s), transcription factor-co-regulator complex(es), or other regulatory proteins involved in transcription, thereby forming regulatory complexes in living cells, are obtained from the cells and analyzed. According to this aspect, the regulatory proteins are preferably cross-linked to or otherwise stably associated with the cis sites or their associated proteins by treatment with reagents that maintain association of the proteins with the nucleic acids or associated proteins through the isolation steps. Cross-linking is preferably achieved by the use of reagents or compounds that allow the subsequent reversal of the cross-links, such as formaldehyde, glutaraldehyde or cleavable linkers. Alternatively, the regulatory proteins can be cross-linked to regulatory regions by a physical means such as UV light or energy at other wavelengths. In all cases, it is preferred that cross-linking reagents, or cross-linking itself, should not affect the ability to detect one, or all, of the components or reactants of the complexes. For both the cell-free and cell-based methods of complex formation, complexes can be isolated from unbound or otherwise undesired reactants by various means known to those skilled in the art and as described herein.

In accordance with the present invention, the cis site-regulatory protein complexes and regulatory protein-regulatory protein complexes are characterized according to the specific types of components that comprise the complexes and how often such components are found to occur in complexes, in order to determine which components are active and to what level they are active in the cell population analyzed. Such characterization is accomplished by any number of methods, including, but not limited to, amplification of specific nucleic acid regions capable of being bound in complexes; sequencing of the nucleic acid molecules or proteins found in the complexes; hybridization of the bound nucleic acid molecules to other nucleic acid molecules of known sequence for identification purposes, identification of the regulatory proteins by biochemical or physical means; isolation and/or purification of the components utilizing affinity reagents; subjecting the components to arrays of molecules for use in identification, or employing other detection systems that allow direct visualization or identification of the cis sites, larger nucleic acid regulatory regions of which they are part, and/or regulatory proteins bound.

In a preferred aspect, the present invention provides a method for characterization of nucleic acid regulatory regions containing cis sites comprising the amplification of regions suspected of being bound, or having the potential to be bound, in such regulatory complexes. Protocols and methods for nucleic acid amplification include polymerase chain reaction (PCR), quantitative PCR (Q-PCR), real-time PCR, ligation-mediated PCR (LM-PCR), rolling circle amplification, transcription-mediated amplification, ligase chain reaction and the like. Protocols and methods for protein amplification include cloning and expression in prokaryotic and eukaryotic cells, de novo protein synthesis for small proteins and peptides, and amplification of cells expressing the proteins followed by protein purification. Such methodologies are practiced by those having skill in the art.

According to this invention, active nucleic acid regulatory regions are also identified by direct sequencing of the nucleic acid, e.g., DNA, fragments that are isolated as a result of being bound by, or otherwise stably associated with, a nucleic acid binding factor, nucleic acid binding factor plus co-regulator combination, or other regulatory protein involved in gene expression. In certain cases, nucleic acid fragments are isolated, amplified using well known amplification methodologies, and then sequenced. Alternatively, the nucleic acid fragments are cloned in appropriate vectors before sequencing. The nucleic acid fragments can also be concatamerized end-to-end before cloning and sequencing. In another aspect, the isolated nucleic acid fragments are used as a template to make a nucleic acid library, which can be size-fractionated to yield similarly-sized nucleic acid sequences. The resulting nucleic acid sequences can then be concatamerized and cloned, and the cloned nucleic acid can be subjected to nucleic acid sequencing. In cases in which the nucleic acid is RNA, it can be reverse-transcribed into DNA using reverse transcriptase and subjected to the same steps as described above for DNA.

In an alternative aspect, the active regulatory regions are identified by hybridization of the nucleic acid fragments isolated from the binding reactions to other nucleic acid molecules of known identity. Other detection systems can be used that allow direct visualization or identification of the nucleic acid sequences bound by one or more nucleic acid binding factors, nucleic acid binding factor-co-regulator combinations, or transcription-associated regulatory proteins. Proteins involved in active regulatory regions are also identified by the use of reagents and methods that specifically recognize particular proteins or portions of proteins. These include, but are not limited to, 1) immunodetection using specific antibodies or portions of antibodies, 2) molecules that bind specifically to other specific molecules and that can be attached to, or inserted into, the regulatory protein so that the specific molecule becomes a tag, and 3) receptor-ligand interactions.

Another aspect of the present invention provides methods of comparing the global gene regulatory element activity profiles from cells comprising two different cell populations and determining which elements exhibit differential activity between the two populations. Such methods comprise comparing the type or quantity, or both, of active cis site-regulatory protein complexes, regulatory protein-protein complexes, or regulatory protein-transcribed region complexes formed from one cell population with the same types of complexes formed from the other cell population. Such a comparison generally involves the activity levels of more than one type of complex in each cell population. In accordance with this aspect, cell populations to be compared comprise different cell types within the same organism, the same cell type between different organisms, normal versus diseased cells of the same type, normal versus transformed cells of the same type, cells at different stages of differentiation or development, cells treated with an exogenous material such as a drug, compound or other molecule versus untreated cells, cells exposed to two different compounds or molecules, cells exposed to a different external or internal condition versus unexposed cells, cells exposed to two different external or internal conditions, or cells comprised of more than two different cell populations (each of these comparisons comprising a comparison of cells in cell populations that represent three or more different cell types, sources, treatments, physiologic and/or metabolic states).

In a related aspect, regulatory element activity profiles obtained for the different cell populations are directly compared in order to determine differences in gene regulatory activity, and hence gene expression, between the two or more cell populations. In a further related aspect, profiles obtained for different metabolic or physiologic states are compared between cell populations (preferably cells of the same lineage) in order to determine differences in gene regulatory activity and gene expression. The comparison of global profiles can also be at the level of a low number of cells including single cells, provided that the sensitivity of detecting multiple regulatory complexes is adequate, for example, by detecting and characterizing the complexes in situ or by amplifying one or more components of the complexes for the purposes of analysis.

Another aspect of this invention involves carrying out the methods of the invention in a sequential or parallel manner in any combination in order to add to the global regulatory element profiling for any cell or cell population. For example, in one cell population, the cell-free method is first carried out and the bound nucleic acid fragments are analyzed to determine which regulatory elements are active in that cell population. Thereafter, antibodies directed against the transcription factors found to be active are used subsequently in the cell-based method to identify the active promoters and/or transcribed regions used by those transcription factors inside cells, i.e., in the living state. In another example, the cell-based method is used to profile the actively transcribed regions in a cell population using antibodies against transcription-associated proteins; isolated regions will include those at the 5′ ends of the genes. Since the DNA regions bound will be long enough to include promoter regions, another cell-based profiling is carried out using antibodies against specific or general transcription factors in order to identify the promoters of those genes. Alternatively, cell-based profiling is carried out first using antibodies against certain transcription factors, and then the complexes isolated are subjected to antibodies against transcription-associated proteins to identify those genes regulated by certain factors and undergoing transcription. Yet another alternative involves using combinations of antibodies against more than one transcription factor or transcription-associated protein. The possible combinations are not limited to those described here, and all results contribute to the global regulatory element profile for that cell or cell population.

In a preferred aspect, viable cells are profiled for regulatory element activity when the regulatory proteins are cross-linked to the cellular nucleic acid. Similarly, if the regulatory element complexes are to be formed in a cell-free reaction, a cellular extract, such as a nuclear extract, of regulatory proteins is obtained from living cells. Cells can be in a non-living state when the regulatory complexes are obtained, as long as the regulatory element complexes analyzed are representative of the cell state for which the profiling is determined. According to this invention, cells can be individual cells, cloned or otherwise homogenous populations of cells, semi- or fully-purified populations of cells, cells in or from tissues, organs, or portions thereof, or cells from whole organisms. Cell populations can be mixtures of cells, for example, a mixture of two or more specific cell populations, whose composition or characterization is known to those skilled in the art.

It is an aspect of the present invention to provide methods for the analysis of the activity of transcription-associated factors, including but not limited to specific transcription factors or general transcription factors of cells. These factors may bind to their cis sites or otherwise transcription-associated nucleic acid sequences only during the process of transcription initiation or transcription progression. Alternatively, they can be bound to their cis sites or associated nucleic acid sequences at all or most times and are involved in active transcription only when another binding event or molecular association occurs, for example, a co-regulator molecule is also bound, other nucleic acid binding factors bind nearby, or a certain combination of regulatory protein bindings takes place.

In another aspect of this invention, transcription factors or co-regulators that bind to the transcription machinery or other components of the transcription process are analyzed to determine which genomic DNA regions are being actively transcribed. Similarly, components of the transcription machinery, including the polymerase enzyme and its co-regulators, are analyzed to identify those DNA regions that are undergoing transcription. These regions can comprise novel, previously unidentified gene sequences. The transcription rates for genes are also quantifiable by analyzing the number of times a particular cis site-containing nucleic acid, e.g., DNA, sequence or an actively transcribed nucleic acid sequence is found in a bound state by a regulatory protein, or a combination of regulatory proteins.

An aspect of the present invention also includes identifying a particular class of regulatory regions bound by a certain nucleic acid binding factor, or types of nucleic acid binding factors, and then profiling the activity of such regions. For example, enhancer regions can be profiled for activity by identifying nucleic acid sequences bound by transcription factors or transcription factor/co-regulator combinations that only bind, or bind predominantly, to that class of regulatory region. Promoter regions that contain specific cis sites can be profiled for activity by identifying sequences bound by the transcription factors that recognize those particular cis sites. Various types of RNAs can be profiled for activity, e.g., those with higher stability inside cells, due to the presence of certain regulatory sequences that may or may not be involved in specific types of regulatory complexes. Regulatory regions that contain combinations of cis sites can be profiled for activity by identifying nucleic acid sequences bound by the two or more nucleic acid binding factors that recognize those cis sites. Complexes are separated away from unbound components and/or other cellular material before partitioning of certain complexes and analysis to identify the components within those complexes. Alternatively, the complexes desired can be obtained without some of the isolation steps mentioned above, for example, the specific complexes desired are removed from the entire cellular or binding mixture.

In another of its aspects, the present invention provides a method involving the placement of nucleic acid molecules comprising known sequences, which may or may not include particular cis sites or other transcription-associated nucleic acid sequences, in locations on a substrate, preferably in an array, such as in discrete tubes, in microtiter wells, on one or more chips, or on a microarray surface. The localized nucleic acid molecules are contacted with protein extracts comprising nucleic acid binding factors, co-regulators and other transcription-associated proteins, followed by analysis to determine which nucleic acid-protein complexes have formed. Thereafter, the specific cis site-regulatory protein complexes, or other regulatory complexes, are detected by appropriate methods. Suitable detection methods include methods that determine when one of the components in the complex, i.e., the nucleic acid sequence or the regulatory protein, is or was in a bound state. These assays can be homogeneous assays, such as using fluorescence polarization or chemiluminescent labels, or may require the separation of bound complexes from unbound components.

Alternatively, specific nucleic acid binding proteins, with or without co-regulators, are placed in locations on an array, and a library of nucleic acid fragments, also with or without a mixture of co-regulator proteins, is contacted with the array to allow complex formation. Related aspects include direct sequencing of the bound nucleic acid molecules (which can comprise DNA or DNA complementary to RNA) and analysis for cis sites or other regulatory regions within the nucleic acid molecules; biochemical characterization of the bound nucleic acid binding factors or co-regulators including those in protein-protein interactions; hybridization to the bound nucleic acid molecules using specific nucleic acid probes with either a separation step to remove unbound components or a homogeneous assay format; other separation methods based on molecular size, such as capillary electrophoresis; and detection using antibodies directed against proteins associated with regulation of transcription or other processes involving gene expression.

In a further aspect, the methods according to the present invention comprise labeling the nucleic acid molecules or regulatory proteins with detectable molecules or “tags” for detection. Suitable tags include, without limitation, fluorescence, radioactivity, enzymes, chemiluminescence, bioluminescence, antigens that can be bound by antibodies, antibodies that can be bound by antigens, nucleic acid oligonucleotides, and other identifier molecules, such as beads or groups, that can be specifically identified.

In yet another aspect, the present invention provides methods performed in a moderate to high throughput format, for example, a format in which more than about 10, and often more than about 100, 1,000, or 10,000 elements are profiled at once. The format includes an array in which either specific nucleic acid oligonucleotides of known or partially known sequences, or combinations thereof, or specific regulatory proteins of known or partially known compositions, or combinations thereof, are positioned at specific locations of the array comprising microtiter plates, slides, gels, columns, microarrays, tubes, particles, or chips. Within each plurality of regulatory elements, individual oligonucleotides or proteins can be located in separate and distinct locations. The format also comprises arrays, microarrays, and the like, or other solid supports, containing detection elements for nucleic acid-regulatory protein complexes, such as antibodies that bind to proteins associated with transcription, translation or certain chromatin structures, or nucleic acid molecules that bind to cis sites.

In other aspects, the present invention is applicable not only to embodiments involving regulatory elements involved in gene expression processes, such as transcription, but also to other uses in which nucleic acid binding factors bind to nucleic acids in a sequence-dependent manner. Such applications involve proteins binding to single-stranded RNA or DNA, double-stranded DNA or RNA, or nucleic acids with modified bases, or involve other types of molecules binding to RNA or DNA. Another application involves the profiling of other molecules that bind to nucleic acid molecules or nucleic acid binding factors. Other cellular processes, in addition to transcription, that utilize specific nucleic acid-nucleic acid binding factor complexes comprise DNA replication, nucleic acid trafficking, DNA repair, RNA translation, RNA splicing, RNA degradation, nuclear organization, recombination, and nucleic acid amplification.

In accordance with the present invention, global gene regulatory element activity profiling is useful for a variety of applications, as follows:

1) The status of gene expression within cells of any cell population can be determined by analyzing which nucleic acid-regulatory protein complexes are detected globally in the cell populations of interest. Complexes that can be detected are most likely to be regulating specific gene expression, and the groups of genes regulated by each complex can be determined. This information can be used to define groups of coordinately-expressed genes that have changed or are different in their expression patterns between two cell populations of interest.

2) The effects of exogenous materials on cells related to activities such as, for example, efficacy, mechanism of action, toxicity and resistance can be determined by comparing the global profiling results between treated and untreated cell populations or between cells treated with a particular exogenous material versus a reference or otherwise known material. Exogenous materials that can affect gene regulatory element activity profiles include one, or a plurality of, test compounds, such as, for example, small organic molecules, small inorganic molecules, lipids, carbohydrates, peptides, polypeptides, mutant or otherwise altered polypeptides, and nucleic acids. Alternatively, a variety of parameters can be screened, for example, different compound concentrations, different times following compound addition, combinations of compounds, effects on different cells types, and the like. In certain cases, a marker gene, such as a gene encoding luciferase or green fluorescent protein (GFP), can be used in a construct whose expression can be regulated.

3) The effects of altering the external or internal environment of cells, such as, for example, growth conditions, maintenance conditions, or toxic conditions, can be determined by comparing the global profiling results obtained from cell populations exposed or not exposed to various conditions; among cell populations exposed to a variety of conditions; or between cells exposed to a particular condition versus a control, or reference, or otherwise known condition.

4) The effects of conditions that place cells under different states, such as stationary versus growth phases, or growth at different rates or temperatures, or in different nutrients can be determined by comparing global profiling results from cells placed under different states with cells under a control, or reference or otherwise known state.

5) Approaches can be developed to alter gene regulation using molecules such as cis sites, nucleic acid binding factors. co-regulator or regulatory protein inhibitors, inducers, agonists, antagonists or analogs. These can also be used to identify and quantify which cis site-regulatory protein complexes are active within the cell population of interest.

6) The sets of coordinately regulated genes that are controlled by the gene regulatory elements found to be active by the global profiling methods according to this invention can be determined using methods including, but not limited to, knocking-in (supplementation, for example, by artificially expressing or over-expressing nucleic acids encoding certain transcription factors, co-regulators or other regulators), or knocking-out (for example, by cis site decoys, antisense oligos to transcription factor or co-regulator RNAs, or RNAi) certain nucleic acid-regulatory protein activities, or direct sequence analysis of the cis site-containing sequences associated with genes of interest.

7) The genes regulated by specific regulatory elements can be studied for their RNA expression patterns via any method typically used for RNA expression analysis (also called RNA profiling), such as hybridization to nucleic acids on microarrays, macroarrays, filters, gels, particles (beads), or in solution, or with amplification methods, such as reverse transcriptase-polymerase chain reaction (RT-PCR).

8) Disease-related gene pathways involving key regulatory elements, such as certain transcription factors or co-regulators, for example, NFκB in inflammation, peroxisome proliferation activator receptor (PPAR), e.g., PPARgamma, in obesity, T-bet in asthma, and Pax2 in renal disease, can be identified and characterized using information gained from global regulatory element activity profiling.

9) The genetic regulatory circuitry comprising the differentially expressed genes and their regulatory elements, can be defined using information gained from global gene regulatory element activity profiling.

10) Novel, previously-unknown gene regulatory elements can be discovered by analysis of the global profiling data, including, but not limited to, analysis of the nucleic acid molecules that bind one or more nucleic acid binding factors, and detection of the nucleic acid binding factors that bind to novel, previously unknown cis sites.

11) Genes encoding novel regulatory proteins can be studied for their transcription levels by quantification of transcription-related regulatory complexes to determine cell populations, i.e., cell types and conditions, in which these regulatory proteins are present. They can also be studied by RNA expression analysis and the results compared.

12) Active gene regulatory elements important in certain cell populations or diseases of interest, such as cis sites, nucleic acid binding factors, co-regulators and other regulatory proteins, as well as the larger regulatory regions including promoters and enhancers of which they are part, can be determined by analyzing the global gene regulatory element activity profiling results for the cell populations of interest.

13) Genes whose gene products can be targeted for the development of therapeutic drugs or biomolecules, or diagnostic or pharmacogenomic markers, can be identified by analyzing the global profiling results in combination with other information to identify the coordinately regulated gene sets, gene pathways and genetic regulatory circuitry. Therapeutic products or diagnostic or pharmacogenomic tests can be developed.

14) With respect to expressed genes, sets of coordinately regulated genes determined by global regulatory element activity profiling can be studied further for expression levels. In addition, sets of genes regulated by the same regulatory elements can be profiled for RNA expression differences using methods such as RNA expression analysis.

In yet another aspect of the present invention kits, e.g., diagnostic or pharmacogenomic test kits, are provided for determining the global gene regulatory element profiles of cells. Such kits can include arrays of various types, such as microtiter or other micro arrays of nucleic acid molecules, for example, those comprising cis sites, or proteins such as nucleic acid binding factors, for determining global gene regulatory element activity profiles, as well as instructions for use.

Further aspects, features, and advantages of the present invention will be better appreciated upon a reading of the detailed description of the invention when considered in connection with the accompanying drawings/figures.

### DESCRIPTION OF THE DRAWINGS/FIGURES

FIG. 1 shows a scheme for profiling regulatory element activity using fluorescence polarization. DNA molecules from a library are placed into individual wells of microtiter plates such that each well contains a unique sequence that is unknown (represented by letters S-Z), or one that is known to bind sequence-specific DNA-binding proteins (e.g., AP-1, NF-κB, OCT-1 or SP-1). When nuclear extract from resting Jurkat cells is added to plate A and nuclear extract from PMA/ionomycin-activated Jurkat cells is added to plate B, a significant increase in the fluorescence anisotropy from the AP-1 and NF-κB cis site-containing DNA molecules in plate B is expected compared to plate A, as a result of known induction of both AP-1 and NF-κB binding activities upon Jurkat cell activation with TPA/ionomycin. In contrast, a rapid and significant increase in the fluorescence anisotropy of the labeled OCT-1 and SP-1 fragments is expected in both plates A and B equally, as a result of the moderately high constitutive levels of OCT-1 and SP-1 binding activities that are found in both resting and activated Jurkat cells.

FIG. 2 presents the results of an electrophoretic mobility shift assay (EMSA) in which nuclear extracts obtained from resting or TPA/ionomycin-activated Jurkat cells were used in separate binding reactions containing a ³²P-labeled oligonucleotide comprising a binding site for NF-κB. As shown in lanes 4 and 6, a significant increase in the gel-shifted material (DNA-protein complexes) from the activated Jurkat cells was observed when no competitor (lane 4) or mismatched competitor (lane 6) was included. In contrast, matched competitor oligonucleotide to the NF-κB site prevented the formation of specific NF-κB complexes (lane 5). Also, no increase in gel-shifted material was observed when the nuclear extract was obtained from resting Jurkat cells, regardless of whether the reaction also contained no competitor (lane 1), matched competitor (lane 2) or mismatched competitor (lane 3). These results demonstrate that NF-κB regulatory complexes are differentially present in Jurkat cells that have been activated.

FIG. 3 presents a graph wherein bars indicate the percentage of DNA fragments containing selected cis sites that were isolated in binding reactions containing nuclear extracts from either untreated (white bars) or NGFbeta-treated (black bars) PC12 cells. The graph shows partial regulatory element activity profiles for both cell populations; other cis site-nucleic acid binding factor complexes were also observed, but not included in the graph. As indicated, the profiles of the two cell states are markedly different from one another.

FIG. 4 represents the results of an electrophoretic mobility shift assay (EMSA) in which nuclear extracts obtained from PC12 cells were either untreated (Control, “CONT”) or treated with NGFbeta in separate binding reactions containing ³²P-labeled oligonucleotide comprising a binding site for a specific transcription factor. As shown in lanes 3 and 4, there is a significant increase in the gel-shifted material (DNA-protein complexes) of the NGF-treated cells when the oligonucleotide was specific for the AP-1 binding site. In contrast, no increase in gel-shifted material was observed when the oligonucleotide was specific for the OCT-1 binding site (lanes 7 and 8). These results demonstrate that AP-1 is differentially activated in the NGF-treated cells, while the OCT1 CIS/Trans complexes are present in both cell populations, but there is no activity differential between the NGF-treated and untreated cells. Lanes 1-2 and 5-6 lanes are from binding reactions that did not include intact nuclear extracts.

FIG. 5 shows a flow chart for global regulatory element profiling according to the present invention.

FIG. 6 shows a nylon filter spotted with single-stranded oligonucleotides containing eight different cis site motifs and then hybridized with ³²P-labeled DNA fragments that had been isolated as a result of nuclear protein binding. Jurkat cells, both resting and activated with PMA and ionomycin, were used as the sources of nuclear protein extracts. Each extract was added to a mixture of ³²P-labeled DNA fragments, each representing a particular cis site motif; namely, AP1, AP2, EGR, OCT, UJI, ETS, XFD and YY1 cis sites. It will be appreciated that cis sites are typically named according to the nucleic acid binding factors that recognize and bind to them, and the factors are named within the art according to certain biological characteristics or other associations, e.g., AP-1 is the shortened version of Activator Protein 1, EGR is shortened for Early Growth Response, OCT is shortened for Octomer Binding Protein, and so on. DNA-protein complexes were allowed to form and then separated, and the labeled DNA fragments were isolated and hybridized to the filter. Significantly greater signals were observed for AP-1 and EGR cis site-containing fragments in the activated Jurkat cells versus the resting cells, indicating increased binding of those transcription factors in the activated cells. These results agreed with those obtained by direct sequencing of the isolated DNA fragments in other experiments. Also, the intensities of hybridization signals on the filter agreed with the percentages of those cis site motifs observed relative to total cis sites counted when the DNA fragments were sequenced.

FIG. 7 represents a polyacrylamide gel showing the detection of DNA molecules that had been immunoprecipitated with antibodies against transcription-related proteins TFIIB, TBP, TBIIEβ, CBP, and AcH3 (acetylated histone H3). Formaldehyde-crosslinked chromatin from both Jurkat (J) and MCF7 (M) cells was immunoprecipitated, the DNA was eluted, and PCR was used to detect specific sequences in each immunoprecipitate. For each pair of gel sections, the name of the antibody used is listed, followed by the gene amplified in those particular PCR reactions. For each gene, the region amplified corresponded to a segment at the 5′ end (in other experiments, regions corresponding to the 3′ ends of genes were also tested and gave similar results). Some genes were found to be transcribed at higher levels in the MCF7 cells (e.g., ER and c-ERB), while other genes (e.g., LEF-1) were transcribed at higher levels in Jurkat cells. The transcription levels of some genes were the same between the two cell types (e.g., histone H3). Some genes exhibited different transcriptional activities, depending upon the transcription-related protein examined, e.g., c-FOS. These results demonstrate the usefulness of regulatory element profiling for identifying DNA regions involved in the regulation of gene transcription.

FIGS. 8A and 8B present portions of polyacrylamide gels showing the comparison of DNA fragments found to be immunoprecipitated with antibody against RNA Pol II (left panel), with steady state mRNA levels detected by RT-PCR (right panel). For the immunoprecipitation, antibody was added to formaldehyde-crosslinked chromatin, DNA was eluted, and PCR was used to detect specific sequences in the precipitate. Names of the genes detected in each reaction are listed to the left of the gel portions. NA=Not available. Within each panel of FIG. 8A, each gel segment represents the signal detected from MCF7 cells (M) or Jurkat cells (J). For each gene where both types of data were available, results from the Pol II immunoprecipitation agreed with the RT-PCR. In FIG. 8B, each gel segment within each panel represents the signal detected from resting Jurkat cells (R) exposed to DMSO for 3.5 hours, or activated Jurkat cells (A) exposed to 1 mM PMA, 2 mM ionomycin in DMSO for 3.5 hours. Again, for almost all genes, DNA transcription levels corresponding to Pol II immunoprecipitation agreed with RNA levels as determined by RT-PCR. One exception was SATB1, where the immunoprecipitation showed higher levels of gene transcription in activated cells, while RT-PCR showed resting and activated cells to be equivalent.

FIGS. 9A and 9B present data generated by quantitative PCR on immunoprecipitated DNA and cDNA from resting and activated Jurkat cells. Resting cells were exposed to DMSO for 3.5 hours, and activated cells were exposed to 1 mM PMA, 2 mM ionomycin in DMSO for 3.5 hours. In FIG. 9A, bars represent relative values for the amount of DNA immunoprecipitated with anti-Pol II antibody. Data were normalized to input chromatin (no immunoprecipitation), and signals generated from “no antibody” controls were subtracted. FIG. 9B: Quantitative RT-PCR was carried out on RNA isolated using Trizol (GibcoBRL). RNA was treated with DNase I to remove contaminating genomic DNA, and cDNA was generated using AMV-RT (Invitrogen) and random hexamer primers. The same gene-specific primer sets were used in both FIGS. 9A and 9B. The signal intensities in (B) are measured on a log scale. Again, most genes that are differential in transcription as shown by Pol II immunoprecipitation are also differential in their RNA levels. Two exceptions include SATB1 and cMYB, which appear differential by gene transcription but unchanged at the level of RNA. SATB1 was also differential by immunoprecipitation followed by PCR and gel analysis, as shown in FIG. 8B.

FIG. 10 presents data generated by quantitative PCR performed using immunoprecipitated DNA from Jurkat and MCF7 cells. Bars represent relative values for the amount of DNA immunoprecipitated with anti-AcH3 antibody (AcH3 represents acetylated histone H3). Data were normalized to input chromatin (no immunoprecipitation), and signals generated from “no antibody” controls were subtracted. Some genes were transcribed at higher levels in Jurkat cells (e.g., HPK, CD3, CXCR4 and ITK), while others were transcribed at higher levels in MCF7 cells (e.g., ER and cERB).

### DETAILED DESCRIPTION OF THE INVENTION

The present invention provides novel methods for performing global profiling of gene regulatory element activity in any cell or cell population and determining differences in gene regulatory element activity between two or more cells or cell populations in order to identify differences in gene expression between and among cells. The comparison of global profiles and the determination of differentially active regulatory elements are used to identify differences between cells at the level of gene expression. Such determinations can yield information about cells with respect to their differentially transcribed genes, as well as differences in cell behavior and function involving growth, viability, differentiation, drug resistance, susceptibility to infectious organisms, production levels of certain gene products, and any other characteristic that can be measured, which is due to, causes, or is associated with a change or changes in gene expression. The effects on cells of drug compounds, bioactive agents, substances, reagents, and the like, using endpoints of, for example, efficacy, toxicity, mechanism of action, and the like, can also be obtained by the practice of the described global profiling methods.

By the practice of the methods of this invention, the transcriptional blueprint of any cells of interest can be determined by examining the individual binding activities of entire populations of proteins derived from the cells (e.g., transcription factors and co-regulator , i.e., transcription-associated proteins) and/or the nucleic acids to which they bind or with which they are in association. Measuring transcriptional regulation and activity can be used as a straight-forward readout of gene expression changes, in that changes in expression of classes of genes are measured, i.e., those that are controlled by specific regulatory elements. Information obtained from the global profiling methods as described herein can be very revealing and instructive without requiring the profiling of thousands of genes, or tens or even hundreds of thousands of RNA transcripts in any cell. The computational power and analysis tools required for RNA profiling are both significant and controversial. Further, transcription is a major level at which gene regulation occurs, and RNA profiling, which measures steady state levels of RNA, does not always indicate transcriptional activity. That is, RNA levels are due to multiple processes besides transcription, including processing, splicing, trafficking, translation, and degradation.

Studying the activity of regulatory elements allows the determination of which nucleic acids, such as specific genes, are regulated together, and the relationships between these genes and between their regulatory elements. Understanding how genes interact with each other and with regulatory elements allows for the analysis of coordinate expression of genes and activities of regulatory elements. Thus, if there is a change or difference between cells, it is possible to understand the multiple points of impact, and then to also identify and characterize the subsequent alterations that occur as a result of the initial changes. Other methods such as RNA profiling provide information only on coincidental expression, and not the coordinate expression of genes.

Definitions

All technical and scientific nomenclature and terms used herein are intended to have the same meaning as is commonly understood by one having skill in the pertinent art. Where a term is provided in the singular, e.g., cell, it is to be understood that the plural of the term, e.g., cells, is also contemplated. The following definitions are provided for guidance and are not meant to limit the present invention in any way.

By “global profile” is meant the activity levels of gene regulatory elements in a cell population as determined by the extent of formation of specific binding complexes involving two or more regulatory components (or “elements”), such as nucleic acid molecules comprising one or more cis binding sites, nucleic acid binding proteins or factors, and/or co-regulatory molecules, including, but not limited to, transcription-associated proteins and factors, e.g., polymerase (pol) enzymes. Complexes comprise interactions between cis binding sites and nucleic acid binding proteins or factors, between cis binding sites, nucleic acid binding proteins or factors and co-regulatory molecules, between nucleic acid binding proteins or factors and co-regulatory molecules, and between co-regulatory molecules. A global profile comprises a collection of activity levels of known cis binding sites, their associated transcribed regions, nucleic acid binding proteins or factors, and co-regulatory molecules, or a portion thereof, in the cell population undergoing analysis. As additional cis binding sites, nucleic acid binding proteins or factors, and co-regulatory molecules are discovered, e.g., while carrying out the methods in accordance with the present invention, they can be added to the gene regulatory element activity profiling analysis. In addition to the activity levels of gene regulatory elements as determined by the extent of complex formation, a global profile also comprises a collection of activity levels of the regulatory elements wherein activity can mean changes in the elements that affect their ability to bind in specific complexes involved in gene expression. By “gene” is meant a particular sequence of nucleic acid, e.g., DNA, in a genome (which can be discontinuous in the nucleic acid) that encodes a particular protein or related group of proteins.

By “gene regulatory element” is meant a molecule that regulates a function or behavior of one or more other molecules involved in the expression of a gene or polynucleotide, where “gene expression” or “polynucleotide expression” refers to the transcription of DNA into RNA and then usually (but not always) the translation of the transcribed RNA into a protein or polypeptide encoded by the particular gene or polynucleotide. Gene regulatory elements, referred to hereinafter as “regulatory elements” comprise cis binding sites, nucleic acid binding factors, and co-regulatory molecules. Gene regulatory elements can include proteins that functionally associate with nucleic acids, e.g., polymerase and other transcription-associated proteins, histones, capping enzymes, histone-modifying enzymes, transferases, splicing enzymes and the like.

“Cis binding site”, also referred to herein as “cis site”, refers to a defined nucleic acid sequence (or sequence motif) that is capable of associating with an endogenous or exogenously supplied nucleic acid binding factor or protein, whereby the specific complex formed is typically used by the cell to regulate a cellular process involving gene expression. Examples of such cellular processes include transcription, RNA processing such as RNA capping and splicing, and translation.

By “sequence motif” is meant a nucleic acid sequence in which some of the nucleotide positions can comprise more than one possible base so that a limited amount of degeneracy, i.e., generally involving 33% or fewer of positions, is allowed within the cis site. Cis sites include members of cis site families where a subset of the nucleotide sequence is identical or closely related among family members. Cis sites are also referred to and understood by alternate terms, including cis acting nucleic acid sites, regulatory sites, gene-specific regulatory elements, and site-specific binding domains. Cis sites can be located within longer nucleic acid regions called regulatory regions, and can comprise single- or double-stranded nucleic acid molecules. Cis sites and the regulatory regions of which they are a part can be located within, proximal to, a short distance from (e.g., within several hundred bases), or a long distance from (e.g., tens or hundreds of kilobases) the nucleic acid regions that they regulate.

By “nucleic acid binding factor” is meant a regulatory protein or regulatory molecule that binds to a cis site or family of cis sites in a sequence-specific manner, whereby the complexes formed in the cell are involved in regulating a cellular process involving gene expression. Nucleic acid binding proteins that are involved in the process of transcription are typically called transcription factors. Other terms understood and used for transcription factors include transcriptional activators and repressors, gene-specific activators and repressors, transcriptional regulator proteins, or activators and repressors. Transcription factors generally bind in a site-specific manner and often recruit other molecules called co-regulatory molecules or other specific transcription factors. Transcription factors also recruit the transcription machinery to initiate gene-specific transcription. Specific transcription factors may regulate particular genes or subsets of genes based on the locations of each factor's cis binding site motif.

In contrast, “general transcription factors” help to regulate a significantly large number of genes (and in some cases, most or even all of them), and are involved in transcription functions common to all or many genes. These proteins include accessory factors in transcription that recognize the conserved “TATA” box and “initiator” sequences present in many or most protein-coding genes and recruit the polymerase to the start site of transcription. General transcription factors are also involved in assembly of the pre-initiation complex (PIC) and/or the transcription machinery, where the PIC comprises polymerase and chromatin-remodeling enzymes, and the transcription machinery comprises polymerase and other proteins, such as those involved with RNA elongation. Certain general transcription factors can be bound to the nucleic acid, e.g., DNA, at every site of transcription initiation. Nucleic acid binding factors also comprise other nucleic acid molecules, including DNA or RNA molecules, e.g., small or micro RNAs that bind specifically to certain RNAs that are involved in gene expression.

By “co-regulatory molecule”, hereinafter referred to as “co-regulator”, is meant one of a diverse family of regulatory proteins that affect the activity of nucleic acid molecules and/or other elements involved in regulating gene expression such as cis sites, the larger regulatory regions containing cis sites, nucleic acid sequences capable of being transcribed, nucleic acid binding factors or other co-regulators. Co-regulators are recruited to regulatory regions by sequence-specific nucleic acid binding proteins, and can be required for regulation of gene expression. They exert their influence by binding to nucleic acid binding factors or other co-regulators. Examples of co-regulators include co-activating and co-repressing proteins involved in the transcription process, generally referred to as co-activators and co-repressors. Other names known to those skilled in the art include transcriptional cofactors, chromatin-modifiers, histone-modifiers, chromatin-remodeling enzymes, chromatin disrupters, and effectors that read and write the histone code. Some transcriptional co-regulators are directly involved with the transcription machinery or comprise components of the transcription machinery. Other examples of co-regulators include proteins involved in pre-mRNA processing. Co-regulators can also comprise molecules other than proteins, including small molecules, heavy metals, carbohydrates, lipids, nucleic acids, hormones, known drugs, peptides, and analogs of the above.

By “regulatory region” is meant a sequence of nucleic acid that comprises at least one cis site capable of associating with an endogenous or exogenously supplied nucleic acid binding factor. A regulatory region can comprise one or more cis sites. Such a region can be upstream, downstream, in the middle of, or nearer to one end of a gene, a protein-coding region, transcribed RNA, or RNA undergoing or with the potential to undergo translation. Regulatory regions can also be located at distant locations relative to genes. Examples of regulatory regions involved in the process of transcription include promoters and enhancers.

Regulatory elements form complexes comprising two or more elements as described above and are generally referred to as “regulatory element complexes”. Complexes are also referred to herein and are understood by those skilled in the art to include terms such as “cis site-nucleic acid binding factor complexes,” “cis site-nucleic acid binding factor-co-regulator complexes,” “cis site-regulatory protein complexes,” “nucleic acid binding factor-co-regulator complexes,” “co-regulator/co-regulator” complexes, or “co-regulator-transcribed region” complexes. Depending on the analysis conducted, some interacting molecules are present in the complexes but not necessarily detected. For example, co-regulators can be bound to a cis site-nucleic acid binding factor complex but they may not be apparent if the analysis is carried out to determine which cis sites are bound by a particular nucleic acid binding factor. The present invention also provides those skilled in the art with methods to determine and identify previously unknown regulatory elements based upon their involvement in the aforementioned types of complexes. These include but are not limited to, previously unknown cis sites, nucleic acid binding factors, co-regulatory proteins or factors, regulatory regions and transcribed regions.

By “regulatory element activity” is meant the binding of regulatory elements in specific complexes that influence or regulate a cellular process involving gene expression. Further, regulatory element activity can mean physical modifications involving particular regulatory elements, e.g., addition or removal of a chemical group, that affects their ability to bind in the specific complexes that are involved in regulating gene expression. For example, a nucleic acid binding factor, in the presence or absence of co-regulatory proteins and depending on the local environment, binds to a cis site in the process of regulating transcription of a particular gene or genes. Such regulatory elements are determined to be active as a result of their ability to form specific complexes comprising nucleic acid-protein(s) or protein-protein interactions under appropriate binding conditions, either in living cells or in a cell-free environment. The activities of such elements can be detected, and in many instances can be quantified, by the extent of their binding together or their potential ability to bind together in specific nucleic acid sequence-dependent and/or protein composition-dependent complexes.

By “active regulatory elements” are meant those cis sites, nucleic acid binding factors, and/or co-regulators that form specific nucleic acid-protein and/or protein-protein complexes that result as a function of a plurality of proteins in or from a cell or a portion of a cell combining with a plurality of nucleic acid molecules under conditions where regulatory elements specifically recognize other elements and bind to them. Complexes can comprise (1) one cis site plus one nucleic acid binding factor, (2) combinations of cis sites and more than one nucleic acid binding factor, (3) combinations of cis sites, nucleic acid binding factors and co-regulators, (4) combinations of nucleic acid binding factors and co-regulators, (5) combinations of co-regulators, and (6) combinations of co-regulators with transcribed nucleic acid regions. Activity of regulatory elements can also be defined by other modifications of the regulatory elements that lead to a change in gene regulation and expression. For example, regulatory regions containing cis sites can become activated as a result of chromatin modification involving, or in the proximity of, the cis sites or the transcribed regions. Changes can comprise physical alterations, such as unwinding of DNA or a shift to a more open structure. Other elements may have a certain moiety or group cleaved from the parent molecule or be otherwise modified and, in the process, can affect gene regulation.

As used herein, “regulate” or “modulate” refers to the ability to turn on or off or to otherwise alter the function, behavior, amount or activity of molecules or portions of molecules (e.g., activate, repress or enhance) involved in gene expression. Regulating a gene (or polynucleotide) refers to the ability to turn on or off, or otherwise alter, the level of transcription of that gene, that is, up-regulate or activate, or down-regulate or repress, transcription. For example, exposure of a cell or an in vitro transcription system to a drug, compound, or differing condition can cause a gene to be up-regulated or down-regulated relative to the basal level of transcription that would otherwise occur without the particular exposure under the same conditions. Other cellular processes involving gene expression that can be regulated comprise RNA processing, RNA splicing, RNA trafficking, protein translation, RNA stabilization, and RNA degradation.

The term “cis binding site” (cis site) refers to a single-stranded or double-stranded nucleic acid, e.g., DNA or RNA, sequence that can be selectively bound by a nucleic acid binding factor to regulate one or more activities or functions of a nucleic acid sequence present in general on the same nucleic acid molecule. A cis site can also be on a different nucleic acid molecule, which then becomes physically or otherwise associated with the nucleic acid sequence it regulates during the time of regulation. As used herein, a cis site is a nucleic acid sequence, e.g., DNA or RNA sequence, that is associated directly with a specific gene, coding region, transcribed RNA or other functional unit, and can be bound by nucleic acid-binding protein(s) that are 1) used by a cell in regulating gene expression, 2) part of the gene expression machinery, or 3) an exogenous or synthetic molecule that serves the function of an endogenous nucleic acid-binding molecule.

### DESCRIPTION OF THE INVENTION AND EMBODIMENTS

Gene regulatory elements comprise cis acting nucleic acid sites (cis sites), nucleic acid binding factors, and co-regulatory molecules (co-regulators) that form specific complexes in various combinations to regulate gene expression involved in all aspects of cell and organismal growth and development, both normal and abnormal. Thus, cis sites and transcribed regions, nucleic acid binding proteins and co-regulators, when specifically bound together in nucleic acid sequence-dependent and protein composition-dependent complexes, comprise an important aspect of the gene regulatory mechanisms that direct cell activities and tissue function, growth, development, pathogenesis, response to infectious agents and other disease states, regeneration and repair by altering, enhancing, and/or reducing the expression of the genes that are regulated by such complexes.

In its various embodiments, the present invention provides methods to identify the regulatory components that control gene expression in a wide variety of normal cell types. The activity of such regulatory components can be both identified and quantified, and the coordinate sets of genes that are controlled can be identified to attain a base-line or reference characterization or assessment of gene expression and regulation. Such a characterization or assessment can be carried out for different types of cells, including those that are exposed to various compounds or external conditions, and the profiles of regulatory elements, as well as the transcribed regions that they control, are compared between the cell types or treatments. By determining what is different (and what is the same) in cells, the mechanisms behind the differences can be understood; ways to alter differences or prevent changes can be devised; and better therapeutics and diagnostics can be developed. The present invention embraces providing methods for correlating phenotype to genotype, which can be utilized ultimately in rational drug design.

Embodiments of the present invention are directed to methods for globally profiling gene regulatory element activity in cells. The methods include isolating a plurality of gene regulatory element complexes formed in cells and identifying one or more of the regulatory elements comprising the complexes or the genes they control by the procedures described herein. A gene regulatory element comprising a complex is also referred to herein as a gene regulatory component.

According to the present invention, the ability to determine which genes are expressed in a cell, to uncover information about gene expression in a cell, and to analyze active transcriptional events in cells is made possible by identifying, quantifying the activity, and/or determining the characteristics of the regulatory elements that control gene expression. As used herein, the term “cell” can refer to a single cell, or more than one cell, such as a plurality of cells, or a population of cells. In certain cases related to nucleic acid binding sites, information about gene expression is also revealed by analyzing the nucleic acid sequences surrounding the binding sites, which may comprise larger regulatory regions such as promoters and enhancers, as well as the regulated gene regions undergoing or capable of undergoing transcription.

Regulatory elements (or components) generally exhibit activity by binding to other regulatory elements to form specific complexes. In addition, some regulatory elements change their activity due to alterations in one or more inherent properties, such as phosphorylation (Decker T, Kovarik P, 2000, Oncogene, 19:2628-37), acetylation and/or methylation state (Kouzarides T, 2002, Curr Opin Genet Dev, 12:198-209; Freiman RN, Tjian R, 2003, Cell, 112:11-7). Consequently, the regulatory elements are then altered in their ability to participate in and/or remain in complexes. This invention embraces a global analysis of transcription events that are occurring in cells by examining at the same time multiple complexes of different types as formed in cells or a cell population, or as formed outside of cells, yet are representative of complex formation inside cells. The global profiling feature of the present invention provides the simultaneous assessment of a wide variety of transcriptional regulatory elements that are active in cells, e.g., the transcription factors AP1, CREB, E2F, AP2, ETS, OCT, TBP, TFIIβ, TFIIE, etc., by means of one method, or by using a combination of associated methods, of analysis. The methods of the present invention are advantageous in the art because the transcriptional regulatory elements being profiled need not be previously known. Indeed, new elements are able to be discovered and profiled globally in cells through the practice of the methods described herein.

In a preferred aspect, obtaining information about the activity of more than one regulatory element complex, i.e., a plurality of complexes analyzed together, or at the same time, in a global sense, is especially useful for understanding the state of gene expression in a cell. The global approach is also useful for comparing the activities of regulatory elements and the expression levels of genes between or among different cells in order to identify basic differences in gene expression between or among the cells. This type of approach also provides information at the molecular level about functional differences between or among cells, as well as the effects of compounds, mutations, or other changes on or to the cells.

One embodiment of the present invention encompasses a cell-free method for analyzing gene expression by identifying nucleic acid-nucleic acid binding factor complexes that form and represent the complexes present in a cell population of interest. In such a method, the types and numbers of each complex formed are quantified to determine the relative abundance of binding for the various types of complexes in that cell population. This information can then be compared between/among different cells. This method includes a first step of obtaining or providing a mixture or library of nucleic acid sequences (e.g., fragments or segments) that may be representative of nucleic acid of a particular genome. The mixture of nucleic acid sequences may exhibit a specified base composition, e.g., comprising defined percentages of some or all four bases, or may contain modified bases or base analogs. The mixture or library can be randomly generated, partially randomly generated, or specifically defined, wherein the nucleic acid sequences are isolated from cells, obtained by cloning, or synthesized ex vivo by chemical or enzymatic procedures, for example.

The method further involves providing a mixture of proteins from a cell or cell population to be studied. The proteins can be subfractionated, partially purified, or specifically synthesized to be representative of a cell's proteins, or part of a cell's proteins. Typically, the mixture is a nuclear or cellular extract. Thereafter, the mixture of nucleic acid sequences and the mixture of proteins are combined under conditions that allow nucleic acid-nucleic acid binding factor complexes to form based on specific recognition and sequence-dependent binding by nucleic acid binding factors so that nonspecific complexes do not form to any appreciable extent, or if formed, are not detected. The complexes are then isolated away from unbound reactants using physical or chemical properties of the complexes and/or the reactants, such as differences in molecular size, charge, composition, certain moieties (e.g., amino groups), solubility, and the like. The selected nucleic acid sequences (e.g., fragments or segments) are further isolated from the proteins that had been bound to them by the use of standard techniques associated with the manipulation of nucleic acids, including protease digestion, organic solvent extraction of proteins, and precipitation of nucleic acids.

Alternatively, the nucleic acid sequences need not be isolated from other reactants, either at the complex stage or from the bound proteins, as long as the nucleic acid sequences that were bound with protein can be specifically detected and analyzed. The nucleic acid sequences are then analyzed for the presence of cis sites, wherein the analysis includes the determination of both the types of cis sites and the number of times each cis site is present within the selected nucleic acid sequences. In this method, the nucleic acid sequence analysis is performed by sequencing all or a portion of the selected, protein-bound nucleic acid sequences using conventional procedures, and then identifying cis sites among the nucleic acid sequences by comparison with a database of known cis site motifs. A cis site motif is preferably the base sequence motif that the binding site for a particular nucleic acid binding factor comprises, including a reasonable amount of degeneracy allowed by each binding factor. Larger nucleic acid regions, combinations of cis sites, and the absence of cis sites can also be determined as a result of the practice of the method.

In the above method, several rounds of nucleic acid-protein binding are preferably carried out in order to select complexes with a higher and narrower range of binding affinities. With each round of selection, the diversity of nucleic acid sequences that are isolated becomes lower, and the range of nucleic acid-protein binding affinities becomes higher. Additional rounds of binding are accomplished, for example, by placing the nucleic acid fragments isolated as a result of being bound by protein in the previous round into another binding reaction containing another aliquot of protein extract. Binding is again allowed to occur in the same manner as in the first round, or in a different manner, e.g., at a different temperature or with the reactants at a different ratio to each other. For example, the concentration of the nuclear extract is typically high relative to the nucleic acid library in the first binding reaction so that each nucleic acid binding factor is likely to be present in excess over its corresponding cis site(s). These reaction conditions ensure that all or most cis sites contained in the library are bound by their factors in the initial reaction. Subsequently, when the bound nucleic acid molecules are isolated from the first binding reaction, amplified and used in a second binding reaction, each is present at a higher concentration relative to the first binding reaction. This is possible because after the first binding reaction, the resulting nucleic acid mixture exhibits a lower complexity and each cis site molecule present is at a higher concentration. An excess of cis sites over each nucleic acid binding factor in the second and all subsequent binding reactions allows the quantification of the protein factors. The nucleic acid sequences are then isolated according to the same steps as in the first round. This process can be repeated for additional rounds of selection to yield quite specific, high-affinity binding complexes. Preferred are two to four rounds of selection.

In an embodiment of this invention, a cis site is a nucleic acid sequence that is bound by a nucleic acid-binding protein that is 1) used by a cell in regulating the process of transcription; 2) part of the transcription machinery; or 3) an exogenous or synthetic molecule that serves the function of an endogenous transcription-related molecule. In another embodiment, cis sites include nucleic acid sequences that occur endogenously in association with genes whose transcription is regulated. Cis sites can be those previously described, e.g., in the scientific literature, in databases or other sources known to those in the art, or those that are novel and detected and analyzed as a result of the global profiling method of the instant invention. Cis sites comprise nucleic acid sequences within promoters and enhancers, as well as other regulatory regions in nucleic acids associated with gene expression. A “promoter” refers to the minimum nucleic acid sequence necessary to initiate transcription of a gene by an RNA polymerase, for example, in eukaryotic cells, RNA polymerase I (which transcribes ribosomal RNA (rRNA) in eukaryotic cells), RNA polymerase II (which transcribes messenger RNA (mRNA) in eukaryotic cells), and RNA polymerase III (which transcribes transfer RNA (tRNA) in eukaryotic cells), or in prokaryotic cells, bacterial RNA polymerase (which transcribes all RNA in prokaryotic cells). By “enhancer” is meant a cis-acting sequence (regulatory region), that may be some distance away and that increases transcription initiation from a eukaryotic promoter.

Cis sites involved in regulating gene expression are found in a variety of different types of nucleic acid regions, as well as at diverse genetic loci. Certain of these cis sites, for example, TATA boxes and DPE elements (Kadonaga, J T., 2002, Exp. Mol. Med., 34:259-64; Berk A J, 2000, Cell, 103:5-8), are found to be associated with a majority of genes and are generally located a short distance upstream (i.e., in the 5′ direction) or downstream (in cases of DPE) of the transcription start site. Cis sites that are bound by general transcription factors can be associated with many, almost all, or essentially all, genes. Other types of cis sites, for example, hormone response elements, are localized within, adjacent to, or even far from the hormone-responsive genes they regulate. Similarly, cis sites for other specific transcription factors such as those in the CREB family or the AP1 family are located within, adjacent to, or even far from the genes they regulate. Some cis sites are very similar in nucleotide sequence to other cis sites and comprise members of the same cis site family. Some cis sites are recognized and bound by more than one nucleic acid binding factor. In addition, some cis site-nucleic acid binding factor complexes exert variable influences in regard to gene expression, depending on which nucleic acid binding factor is bound to the particular cis site. (Reviewed in Lemon and Tjian, Genes Dev. 14:2551, 2000; Davidson, “Genomic Regulatory Systems,” San Diego: Academic Press, 2001; Orphamides and Reinberg, Cell, 108:439, 2002).

Other embodiments of the present invention encompass methods, preferably performed in solution or on a solid surface, involving different forms of analysis of the nucleic acid sequences obtained from the complexes. In one such method, the nucleic acid sequence analysis is carried out by hybridization of the selected nucleic acid sequences (as a result of being protein-bound) to other nucleic acid sequences which are known to contain cis site motifs, and then observing which of the known sequences form hybrids. More specifically, in the method of this embodiment, the nucleic acid sequences comprising the binding reaction are pre-labeled with a detectable tag, such as a radioactive molecule, an enzyme, a fluorescent molecule, or a chemiluminescent molecule. Alternatively, the nucleic acid sequences may be labeled with a detectable tag after being selected in the binding reaction. The mixture of nucleic acid sequences can be a library of diverse sequences, in which the individual fragments within the library may or may not contain cis sites. Alternatively, the nucleic acid sequence mixture comprises defined sequences that contain known cis sites. Known cis sites sequences are obtained from publicly available databases, such as MatInspector (Genomatix, Germany) or Transfac (Biobase, Germany), from the published scientific literature, and/or from nucleic acid-protein binding information gained using the methods of the present invention.

After binding and complex formation occur between the labeled nucleic acids and cellular proteins, the complexes are isolated from unbound material, and the nucleic acid sequences are separated from the proteins to which they were previously bound. The isolated nucleic acid sequences are then denatured (if originally double-stranded) and hybridized to single-stranded nucleic acid fragments of known sequence under moderate stringency conditions (for example, 42° C., 5×SSPE, 16 hr) followed by washing at high stringency (for example, 0.3×SSC, 65° C.). These fragments of known sequence are distinguishable, e.g., by location or label, in order to determine which form hybrids with the mixture of isolated nucleic acid sequences. For example, the known, single-stranded fragments can be situated or placed in individual tubes, or wells of a microtiter plate; or on a macroarray, such as a nylon filter; or on a microarray. By determining which of the nucleic acid molecules isolated as a result of being protein-bound hybridize to which nucleic acid fragments of known sequence, the sequences of the nucleic acid molecules and the cis site motifs that they contain can be determined. Also, the intensity of signal from the detectable label, which is indicative of the number of hybrids formed, indicates the number of complexes formed in the binding reaction.

In yet another method embodied by the present invention, the nucleic acid-nucleic acid binding factor complexes are formed in specific locations (e.g., in individual solutions or on localizing surfaces), such as on solid substrates, so that individual types of complexes can be detected and quantified. This method involves placing or localizing a first type of reactant, or reactant mixture, i.e., either the nucleic acid sequences or nucleic acid binding proteins or factors, in individual locations, such as in solutions with known locations or on a localizing surface. Nucleic acid or protein molecules immobilized on a localizing surface are stably attached by employing standard techniques, for example, by drying, UV-crosslinking, and the like. The localizing solutions or surfaces comprise individual tubes, beads, particles, wells in a microtiter plate, glass slides, membranes, filters, macroarrays, and microarrays commonly referred to as chips. Thereafter, a second reactant or reactant mixture is contacted with the first reactants in all of the locations under conditions allowing binding to occur between the components of the first and second reactants, or reactant mixtures, and detecting which locations contain bound complexes. Complexes can be detected without isolation from unbound reactants, or they can be isolated from unbound reactants and then detected. This method is advantageous because it is amenable to high throughput analysis involving large numbers of regulatory element reactants.

The detection of complexes is carried out using one of a variety of methods, which can vary depending on whether the nucleic acid or the proteins are originally contained within or on the array. In the case of nucleic acid arrays, in which the nucleic acids are either double-stranded or single-stranded, nucleic acid sequences can be labeled with a fluorescent tag and the complexed nucleic acid molecules detected by fluorescence polarization. Alternatively, the immobilized nucleic acid sequences can have other attached (conjugated) detector molecules that become altered as a result of protein binding, for example, molecular beacons that emit a different signal as a result of binding (Heyduk and Heyduk, 2002, Nat. Biotechnol., 20:171), or chemiluminescent molecules that change in their properties when bound, e.g., acridinium ester (Arnold et al., 1989, Clin. Chem., 35:1588). Since this type of array allows the binding and immobilization of specific proteins at defined locations, antibodies that recognize the bound proteins can be used to identify specific nucleic acid-protein complexes. Antibodies or other affinity reagents can also be used to identify certain classes of nucleic acid molecules or nucleic acid-binding proteins (or factors).

In those cases in which the nucleic acid binding factors are in known locations, the nucleic acids that bind and complex to these factors can be detected by the hybridization of other labeled nucleic acids acting as probes. Such probes are generated by standard methods known to those in the art, most typically by synthesis using nucleic acid synthesizing machines, or by isolation of cloned or cellular genomic DNA. The probes are also typically labeled with an appropriate tag to allow detection of hybrids, including radioactive tags, enzymes, fluorescent or chemiluminescent labels, or any other molecules that can be identified and/or quantified.

Another method embodied by the present invention comprises the isolation of regulatory element complexes that form inside cells. The complexes can be analyzed directly inside of the cells, or isolated from cells (obtained away from intact cells) to generate a global regulatory profile, or to add to a regulatory profile or partial profile that has been generated by another embodiment of this invention. When analyzing directly inside of the cells, profiling can be carried out using a very low number of cells, even down to a single cell. To facilitate the isolation of nucleic acid-nucleic acid binding factor complexes in accordance with this method, it is advantageous to link together the components of these complexes prior to their isolation to ensure sensitivity and specificity of the complexes. It is advantageous, but not necessary, to know at least some of the components in the complexes. Cross-linking is accomplished by a number of suitable methods, for example, physical methods, such as UV light, chemical methods such as treatment with formaldehyde or other “fixatives”, or the use of specific linkers that tether together the various physically-associated molecules. Linkages can be covalent or non-covalent, and are either reversible or irreversible. Reversible linkages are preferred.

Cells are lysed or opened by standard treatments, such as exposure to detergents, other reagents that produce holes in membranes, and/or changes in ionic strength or tonicity, or by physical means, e.g., pressure, force, enzymes, heating, freezing/thawing, electroporation, and the like. The crosslinked nucleic acid-protein complexes from the cells are then treated so that the nucleic acid molecules are sheared or cut into smaller pieces. Various methods can be used to cut the nucleic acids, including sonication, restriction enzyme digestion, limited nuclease treatment, other physical methods such as pressure and heat, and the like. The nucleic acid-protein complexes can be purified or partially-purified from unbound cellular components before cleaving the nucleic acids. Alternatively, the nucleic acid-protein complexes can reside within a mixture or lysate containing other cellular components. A preferred method according to this invention involves obtaining the nucleic acid-protein complexes in a cellular lysate, without further isolation or purification, and then using sonication and buffer conditions that shear the nucleic acids into fragments of approximately 200-1000 bases or base pairs.

Specific complexes containing certain components of interest are then isolated from the rest of the mixture using molecules that bind to those specific components or that take advantage of properties of those specific components. For example, antibodies that recognize certain nucleic acid binding proteins are preferably used. Antibodies that recognize epitopes or structures of proteins that bind to nucleic acid-binding factors are also used. In addition, reagents that recognize and bind to particular epitopes or structures that are themselves attached to certain components of the regulatory complexes can be used. Examples of such reagents include members of receptor-ligand pairs, or other known, interacting reagents or molecules, such as biotin-avidin or biotin-streptavidin. As described further herein, fragments or portions of antibodies, such as single chain antibodies or intrabodies are also useful for isolating complexes. Preferably, the complexes are isolated as a result of the component proteins involved in the complexes, although it is also possible to isolate certain complexes based upon their nucleic acid compositions or sequences.

The selection of regulatory elements can be accomplished by detecting and binding to a wide variety of molecules specifically involved with gene expression. In the case of transcription, useful molecules include transcription factors such as OCT, CREB, AP1, AP2, E2F, ER, or one or several of the hundreds of factors known to those in the art. Selection may also utilize factors known as general transcription factors, which are factors more generally involved in the transcription process, such as TFIIB and TFIIE. Other transcription-related proteins include histone-modifying enzymes, such as acetylases, deacetylases, methylases, demethylases, kinases, phosphatases, and phosphorylases, or the proteins they specifically modify, for example, histone H3 (or its acetylated or otherwise modified version), histone H1, histone H4, and the like. Proteins (factors) that are associated with certain types or classes of promoters or enhancers can also be used for selection; these include factors such as CBP (CREB-binding protein). Other molecules involved with or associated with transcription such as RNA polymerase, elongation factors, or RNA processing factors, such as those used in mRNA capping, can also be used for selection of regulatory elements involved in gene expression.

Following isolation of the desired, specific nucleic acid-protein complexes, the individual components of those complexes are analyzed. Preferably, the nucleic acid molecules, or fragments thereof, comprising the specific complexes are analyzed. To this end, fragments are cloned into nucleic acid vectors using conventional recombinant DNA methods, or they are analyzed directly. Nucleic acid fragments or portions thereof isolated as a result of being bound by a regulatory protein or proteins, or as part of a regulatory complex, can also be amplified using polymerase chain reaction (PCR), ligation-mediated PCR, transcription-mediated amplification, or other amplification methods to generate nucleic acid fragments specific for particular genes or intergenic regions, or for entire populations of fragments that are analyzed to discover which sequences are present in the population. In this case, amplification is carried out in order to provide enough copies of the particular fragments for detection.

Amplification may also be carried out at a limited level, e.g., PCR amplification for 10-15 cycles, in order to provide more copies of the selected fragments for subsequent amplification and detection. In this aspect, the amplified fragments are analyzed by gel electrophoresis, using either non-radioactive detection or after incorporation of radioactive precursor bases or nucleotides. Alternatively, the amplified fragments can be hybridized to macro- or microarrays of known nucleic acid sequences in order to identify which fragments were present in the selected complexes. In yet another type of analysis, direct sequencing of the nucleic acid fragments is carried out, followed by evaluation of the sequences relative to their base composition or base sequence, or to known databases, or to other sources of information, such as the known sequence in the appropriate genome. In a further type of analysis, the nucleic acid fragments are exposed to beads attached to a cDNA library from the cells of interest so that all fragments containing exonic regions will bind to the beads. After washing away all non-hybridized DNA molecules, the hybridized fragments are eluted from the beads, amplified using conditions known to those in the art, and analyzed. The aforementioned steps allow another level of purification that reduces background and increases sensitivity.

In a preferred embodiment, the nucleic acid molecules isolated as a result of binding are used as templates to synthesize a library of short fragments (e.g., approximately 25-100 base pairs in length) by PCR using random primers or by ligation-mediated PCR. By synthesizing shorter fragments that are at least 20-21 base pairs in length, each is highly likely to be unique in the particular genome from which it was derived (unless part of a repetitive element). Further, the shorter fragments can be thought of as samples of the larger fragments from which they were derived, and as samples are much faster to sequence. Preferably, the short fragments are concatamerized into chains of about 10-20 fragments, which affords very efficient sequencing, genomic mapping, and analysis relative to the entire population of sequences in the original isolated nucleic acid. Therefore, each isolated nucleic acid fragment can be sampled via synthesis of a shorter segment that is long enough to map as a unique sequence in the genome.

In a related embodiment, the nucleic acid fragments isolated as a result of binding are ligated with adapters containing sites recognized by type IIS restriction enzymes. These enzymes, exemplified by Mme I, cleave double-stranded DNA at sites approximately 16-20 base pairs away from the recognition site. Adapter-ligated DNA molecules are digested with the appropriate type IIS enzyme, subjected to another ligation to form mixed dimers between the cut ends, digested with a second enzyme that cleaves another site in the adapter, and then used in concatamerization reactions to form chains of approximately 20 fragments of 20 base pairs each (Velculescu et al., 1995, Science, 270:484). The concatamers are then sequenced, analyzed and mapped as described above.

Analysis is also carried out using nucleic acid hybridization in conjunction with specific nucleic acid probes (or targets) to determine which of the molecules that were previously bound by protein form hybrids with the probe (or target) nucleic acids. Hybridization is carried out in any number of formats, including in solution or on solid surfaces, such as on filters, membranes, or microarrays, and quantified by intensity of detectable signal from whatever label is used.

In another preferred embodiment, the selected fragments are detected and quantified by use of a method called real-time PCR or quantitative PCR (Q-PCR). In this method, an aliquot of the selected fragments is placed in contact with amplification primers specific for the two ends of a genomic region suspected to be present in the selected nucleic acid fragments. Amplification is carried out under conditions that allow identification and quantification of the original nucleic acid sequences in the selected mixture, using techniques that are well understood in the art. Such conditions include quantifying the incorporation of labeled nucleic acid precursors or other molecules specific for amplicons over time, such as SYBR green (Becker et al., 1996, Anal. Biochem., 237:204). Alternatively, the Taqman reaction (Roche Molecular Biochemicals, NJ) can be used in which degradation of a 3^{rd} and internal primer is quantified over time, thus indicating the level of amplification accomplished by comparing amplification levels between different samples, e.g., unknowns and standards. Relative amounts of starting nucleic acids in the unknown samples can thus be determined. Alternatives include molecular beacon systems that involve a 5′ fluorescent label and a 3′ quencher. The probe is designed to form a stem-loop structure so that when the quencher is in close proximity to the fluorophore, a low level of fluorescence results. Upon hybridization, the fluorophore and quencher are separated, which results in high fluorescence (Molecular beacon technology is licensed to Public Health Research Institute, Newark, N.J. 07103).

In another preferred embodiment, the selected nucleic acid molecules from two (or more) different cell types, or from differently treated cells, are “subtracted” from each other by methods well known in the art (Konietzko and Kuhl, 1998, Nucl. Acids Res., 26:1359-61; Straus and Ausubel, 1990, Proc. Natl. Acad. Sci. USA, 87:1889-93; Sagerstroem et al, 1997, Ann. Rev. Biochem., 66:751-83). Subtraction is carried out using nucleic acid molecules that have been selected due to being bound by a nucleic acid binding factor and/or coregulator. Typically, two populations of selected nucleic acid molecules obtained from different cell populations are subtracted against each other in order to remove sequences in common. Nucleic acid populations can be amplified, e.g., with PCR, prior to subtraction, after subtraction, or both before and after subtraction. For subtraction, the nucleic acid molecules are tagged by ligating to their ends different double-stranded nucleic acid “adapters”. Nucleic acid (e.g., DNA) fragments from one cell population are preferably tagged with a modified, e.g., biotinylated, adapter. The two nucleic acid samples are then mixed at various ratios, denatured and allowed to anneal under various conditions and for various lengths of time. In a typical, exemplified reaction, the biotinylated selected fragments A are present in 2-10-fold excess over the unbiotinylated fragments B. After hybridization, nucleic acid sequences that are only or predominantly present in B will re-anneal to themselves or remain single stranded, but nucleic acid sequences that are equally present in both samples will mostly form A/B and A/A duplexes. A/B and A/A duplexes are removed, e.g., by binding to streptavidin-coated beads. The unbound nucleic acid fragments are enriched in genomic DNA sequences that are predominantly present in sample B. The procedure is preferably performed more than once to achieve better enrichment of the desired sequences. This method allows the identification of genomic sequences that are differentially bound by a specific regulatory protein or component of the transcription machinery.

Hybridization methods may also be used to select sequences in common between two populations of nucleic acid molecules isolated as a result of being bound by a gene regulatory protein. For example, nucleic acid molecules selected by use of an antibody against a transcription-associated protein such as polymerase are annealed with RNA molecules (or their complementary DNA molecules) to isolate those molecules corresponding to transcribed exons. Nucleic acid molecules selected by use of an antibody against a general transcription factor may be annealed to molecules selected by an antibody against polymerase in order to isolate 5′ends of genes that are transcribed. Nucleic acid molecules selected separately by use of two antibodies against two specific transcription factors may be annealed to isolate sequences regulated by both transcription factors. This aspect, as well as the subtraction approach, can be applied to any combination of isolated molecules or chromatin, and the invention is not limited to those listed here.

Following selection, e.g., by immunoprecipitation, the isolated DNA can be used in any number of applications as mentioned above. Since the amount of DNA resulting from an immunoprecipitation reaction is limiting (typically 5-20 ng of DNA/immunoprecipitation), it is useful to amplify the resulting DNA in order to provide enough material for any application and for an unlimited number of analyses. DNA amplification can be accomplished by ligation-mediated PCR (LM-PCR), wherein known adapter sequences are ligated onto the ends of the blunt ended DNA. Following ligation, primers complementary to the ligated adaptors are used in PCR reactions and the DNA is exponentially amplified. Alternatively, an amplification approach can be used that incorporates the use of adaptors containing the T7 polymerase transcription start site. With this method, a transcription reaction is performed using the immunoprecipitated DNA as a template. The RNA generated in this reaction is then converted to cDNA with the enzyme reverse transcriptase. The cDNA is then used in any of the above applications.

After the sequences are determined for the isolated fragments, the fragments are categorized according to the nucleic acid binding sites, and frequency thereof, that they contain. For example, if the selected fragments, or a portion of the selected fragments, are sequenced, the nucleic acid sequences are analyzed for the presence of known nucleic acid binding factor motifs or known gene sequences. This is carried out by visual observation and recognition, as well as by search functions using computers or other instruments, or by computer programs such as those that search, align or cluster sequences. For computer programs, nucleic acid sequences are searched against databases of known binding sites, such as Transfac by Biobase (Braunschweig, Germany). Examples of programs that carry out search functions include MatInspector by Genomatix (Munich, Germany) and Match by Biobase. Other programs for discovering recurring motifs in nucleic acid sequences include MEME3 (San Diego Supercomputer Center), Gibbs Motif Sampling (GMS) and AlignACE (Roth et al, 1998, Nature Biotech.,16:939). Programs that allow searches of genomic sequences for genes include the Human Genome Browser and the Mouse Genome Browser from the University of California, Santa Cruz, and the Ensembl Genome Browser from the Sanger Institute, Cambridge, England.

In each analysis, known binding sites are identified within the selected fragments which are then catalogued according to the types of sites, number of times detected, and their locations relative to genomic annotation. The number for each binding site or binding site motif is converted to a percentage of the total number of fragments analyzed, in order to normalize the values across multiple cell populations.

In the embodiments in which the selected fragments are analyzed by hybridization, the relative amount of each type of fragment in the selected fraction is quantified by hybridization intensity, particularly compared to hybridization standards that contain known numbers of fragments. In the embodiment in which the complexes form in a format whereby individual components of one of the starting mixtures are placed in certain positions, e.g., arrays, quantification is again determined by the intensity of signal emitted from that location of the array and by considering the binding conditions. In all cases, the information obtained and compiled from the method thus comprises a global regulatory element profile for each type of cell under study.

The regulatory element profile includes both the types of regulatory element complexes found to be active, as well as their relative numbers or intensities of signal as a means to quantify their activities. Results are expressed as percentages of the total in a list, normalized numbers, or relative intensity values, and may be expressed graphically, e.g., using a bar graph format. Profiles can also contain information regarding genomic location within the appropriate genome, preferably the human genome. Mapping data include information such as chromosome number and arm, chromosome band number, or relationship to a genomic marker or markers, such as gene exons, introns, promoters, enhancers, cis sites, repetitive sequences, splice sites, CpG methylation islands, centromeres, telomeres, other known fragments or sequences, and/or nucleotide number in the genome.

Regulatory element profiles comprise the types and levels of activity for the nucleic acid cis binding sites (or the larger fragments in which they are located), the nucleic acid binding factors that recognize and bind to them, any other regulatory factors involved such as co-regulators, and/or the regulated (transcribed) gene regions. Regulatory element profiles can also include RNA levels for transcripts encoding regulatory proteins. Profiles can include the RNA levels for genes specifically controlled by the regulatory elements associated with the genes, or discovered or identified according to the present invention.

Another method embodied by the present invention involves a comparison of profiles from different cells or cell populations comprising the use of either the cell-free binding method or the method wherein binding is accomplished in intact cells or a combination of the two methods, as described herein, in order to determine a regulatory element profile for each of the given cell types or populations of cells.

The global profiling methods of the present invention advantageously provide an analysis of cellular events involving gene expression that allow a “big-picture” analysis of the regulatory molecules and genes that are involved in active transcription in cells. Information can also be gained on classes or groups of genes that are co-regulated and co-expressed, as well as classes of regulatory elements that control one or multiple groups of genes. The information provided by the methods described herein is also dynamic because the global profiling is well suited to comparison of any two or more cells or cell populations, e.g., at different time points or at different stages, so that changes in gene expression and gene regulation are identified. Transcription is a major level at which genes are regulated, and changes in gene transcription, both in terms of which genes are expressed and the levels at which they are expressed, are generally significant and even diagnostic as they pertain to cellular behavior or the effects of external or internal influences on cells.

In contrast, RNA profiling or RNA analysis typically measures steady-state levels of RNA, which provides merely a static picture of the level(s) of RNA transcript(s) in cells. If transcription is occurring at a rapid rate and one or more mRNAs are translated and degraded at a rapid rate, the steady-state level of that RNA will likely be low and its significance can be missed.

Regulatory proteins comprising the complexes can also be isolated and purified or semi-purified, and then analyzed. In one embodiment, the proteins that participate in regulatory complexes are separated away from unbound proteins, isolated using protein methods known to those skilled in the art, and then analyzed using standard methods such as peptide mapping, sequencing and characterization. In another embodiment, specific nucleic acid sequences are used to pull out the nucleic acid binding factors that bind to them, and/or other molecules that bind to the nucleic acid binding factors, resulting in at least a partial purification of the proteins. These proteins can be analyzed by sensitive mass spectrometry methods in order to identify those that are involved in certain regulatory complexes.

The methods in the present invention for profiling regulatory element activity can also be combined with other methods that measure aspects of gene expression in order to construct even more detailed and/or informative gene regulatory element and gene expression profiles. Other methods include RNA analysis (also referred to as RNA profiling); proteomic studies involving gene regulatory proteins; specific assays for transcription factor characteristics, such as phosphorylation; other types of RNA analysis, such as splicing or other processing steps; and the like. With these combined approaches, this invention provides the ability to capture information on virtually all transcribed genes and ongoing regulatory events that involve gene expression in any cell or cell population. With these methods, changes involving disease initiation and/or maintenance, as well as the effects of drug compounds on cells, are just a few of the major areas that can be more clearly elucidated, which can lead to improvement and development of diagnostics, screening tools such as biomarkers, and therapeutics.

The invention described herein provides two avenues for forming the specific regulatory element complexes that are analyzed to determine a global regulatory element profile for any cell population. The first avenue comprises forming the complexes outside of the cell, i.e., in cell-free binding reactions, to regenerate complexes that are formed inside of cells. In the second avenue, the complexes are formed naturally inside of living cells and then are either analyzed inside the cell, or are isolated, and optionally substantially purified, and then analyzed. In either avenue, active gene regulatory elements are identified by detecting and analyzing the specific regulatory complexes that are formed, thereby resulting in a regulatory element profile for that cell type or population. Regulatory element profiles for different cells or cell populations are then compared to each other to determine those activities that are different. These differential activities provide information about differential gene expression and regulation to explain phenotypic differences between the cells being compared, or to determine the effects of various intracellular events or extracellular influences on gene expression in the cells.

In another of its embodiments, the present invention provides methods involving the use of cis sites, which comprise a diverse population of nucleic acid molecules. The term “diverse population of nucleic acid molecules” refers to a composition comprising a plurality of different isolated polynucleotide (nucleic acid) molecules that potentially contain cis sites. The diverse population of nucleic acids used in the methods of the invention can be of a variety of different types, sequences and structures, for example, hairpin structures. The choice of nucleic acid type, sequence and structure will depend on the needs of the methods used to perform the global profiling as well as the desired results to be obtained from such profiles. For example, the diverse populations of nucleic acids of the invention include double-stranded or single-stranded DNA or RNA, as well as linear, circular, or branched nucleic acid molecules. Nucleic acid molecules include those found in nature inside cells and comprise total genomic DNA or RNA or a portion thereof. Nucleic acid molecules of interest can be inserted into standard cloning vectors such as plasmids or viral genomes, or can be connected to linkers or primer binding sites, employing conventional methods and protocols.

In another embodiment, the methods of the invention employ a library or libraries of nucleic acid molecules. Accordingly, the library(ies) comprise a population of nucleic acid molecules containing known cis sites that bind nucleic acid binding factors. Alternatively, the library(ies) comprise nucleic acid molecules that may or may not contain cis sites that bind nucleic acid binding factors. Preferably, the nucleic acid molecules or oligonucleotides used in the methods according to this invention will each contain at least one cis site. In certain embodiments, the nucleic acid molecules comprise 2, 3, 4, 5, 6, 7, 8, 9, 10, or>10 cis sites. Each nucleic acid molecule can contain a different cis site, or some cis sites can be shared among multiple nucleic acid molecules. Such nucleic acid molecules can also comprise defined nucleic acid sequences.

Another embodiment of the invention includes nucleic acid molecules that comprise a genome or can be representative of a genome. In a further aspect, nucleic acid molecules comprise nucleotide sequences found in genomic DNA or cDNA (complementary DNA to RNA). A “defined nucleic acid sequence” refers to a specific sequence of contiguous nucleotides, and is typically represented in the 5′ to 3′ direction using standard single letter notation, where “A” represents adenine, “G” represents guanine, “T” represents thymine, “C” represents cytosine, and “U” represents uracil (in RNA). It will be appreciated that a nucleic acid molecule having a defined nucleotide sequence allows more than one nucleotide type at certain positions, i.e., is degenerate at those nucleotide positions, with respect to one or more positions in the particular sequence. Degenerate nucleotides are represented by any suitable nomenclature, for example, that which is described in World Intellectual Property Organization Standard ST.25 (1998), Appendix 2. Nucleic acid molecules can also comprise the same bias for nucleotide representation as a genome found in nature, for example, A-rich molecules as found in the HIV viral genome or C-rich molecules as found in the HTLV-1 viral genome (Kypr et al., 1989, J. Biochim. Biophys. Acta., 1009:280).

Nucleic acid molecules can be synthetic or isolated from cells, varying in length from about 4 to about 1000 nucleotides in length, or longer than 1000 nucleotides in length, and can comprise purified DNA or RNA, partially-purified DNA or RNA, or unpurified DNA or RNA. Nucleic acid molecules can also comprise DNA within chromatin, a chromosome, or chromosome segment, or can comprise RNA within ribonucleoprotein. In another embodiment of the invention, nucleic acid molecules are those found naturally in living cells and can be of the length and composition found in nature. Suitable nucleic acid molecules are representative of or a part of a genome comprising human, mammalian, vertebrate, invertebrate, animal, plant, fungal, yeast, eukaryotic, prokaryotic or viral genomes. Nucleic acid molecules can contain modified nucleotides, for example, methylated nucleotides, as well as, or alternatively, nucleotide analogs and derivatives. Nucleic acid molecules can also comprise a first amplification primer site upstream of a cis site and a second amplification primer site downstream of the same cis site.

A population of different nucleic acid molecules can be prepared, obtained, or isolated, of any diversity that is appropriate for a particular application of the method in accordance with the present invention. For example, a population of nucleic acid molecules of low diversity can contain 2, 3, 4, 5, 6, 7, 8, 9, 10, 10-20, 20-80, 80-100, or 80-200 different nucleic acid molecules. For certain applications of the invention, it may be preferably to have a population of nucleic acids of moderate diversity and containing, for example, about 200-10³, preferably greater than 10⁴, and more preferably greater than 10⁵ different nucleic acid molecules. In one aspect, if desired, it is possible to synthesize a population of nucleic acid molecules of high diversity, using methods known to those having skill in the art. A high diversity population of nucleic acid molecules contains, for example, about 10⁶-10⁸ different nucleic acid molecules, preferably between about 10⁹-10¹² different nucleic acid molecules, and more preferably about 10¹³-10¹⁵ different nucleic acid molecules.

Pluralities of nucleic acid molecules, e.g., DNA molecules useful for global profiling and that comprise regulatory elements, can be obtained in various ways. For example, nucleic acid molecules can be double-stranded oligonucleotides that comprise nucleotide sequences derived from genomic sequences. Such genome-representative oligonucleotides typically comprise about 25-200 base pairs, preferably 35-100 base pairs, even more preferably 45-50 base pairs of DNA, flanked by primer binding sites. In some cases, the genome-representative sequences may be shorter, that is, between 10 and 25 base pairs. In other cases, the genome-representative sequences may be between 200 and 1000 base pairs, or may be longer than 1000 base pairs. The genomic libraries can also contain short regions of actual genomic DNA, including all functional regions of the genome, typically comprising about 25-200 base pairs, preferably 35-100 base pairs, even more preferably 45-50 base pairs of genomic DNA, flanked by primer binding sites. Genomic DNA libraries can be generated by techniques such as random cleavage of genomic DNA or cleavage by restriction enzymes, followed by cloning into vectors so that the inserts are flanked by both restriction enzyme sites and amplification primer sequences. They can also be synthesized using the genomic DNA as a template, for example, by PCR along with random primers, resulting in short DNA product molecules representative of the genomic DNA. Again, the nucleic acid fragments are preferably constructed so that the inserts are flanked by both restriction enzyme sites and amplification primer sequences, and can be cloned into vectors.

In a preferred embodiment, at least one of the oligonucleotides comprising a duplex is biotinylated at either its 5′ or 3′ end, thereby allowing either the biotinylated oligomer or even the duplex to be detected and/or extracted with streptavidin. In certain alternative embodiments, the region between the primer binding sites comprises a known cis site or a nucleotide sequence that may or may not contain a cis site. For example, oligonucleotides containing sequences 25-200 base pairs in length, preferably 35-100 base pairs in length, flanked by primer binding sites and labeled with biotin at one end, can be employed. Chemical methods for attaching the detectable label biotin (i.e., biotinylating) are known in the art. See, e.g. Agrawal, Chapter 3 in Protocols for Oligonucleotide Conjugates, Volume 26, Humana Press, Totowa, N.J. 1994, pages 93-120 (see especially pages 108-109) and Chu et al, Chapter 5, Id., pages 145-165 (see especially page 157). Oligonucleotides and other nucleic acids can also be biotinylated using enzymatic systems such as, e.g., nick translation (E. coli DNA Polymerase I and DNase I; Boyle, Section V of Chapter 3, in Short Protocols in Molecular Biology, Second Edition, Ausubel, et al. Editors John Wiley & Sons, New York, 1992, pages 3-41 to 3-44) or “tailing” reactions using terminal deoxynucleotidyl transferase (see, e.g., the LABEL-IT™ 3′ Biotin End Labeling Kit from CPG, Inc., Lincoln Park, N.J.).

The nucleic acids useful in global profiling include those already known in the art, for example, all known cis site elements and described in public databases, including binding sites for known transcription factors such as AP1, CREB, NF-κB, E2F, ETS, GATA, HOXF, AP2, NFY, MYOD, OCT, STAT, CEBP, PAX1, COUP, EGR, NHF1, MEF2, NFAT, PBXF, SP1, STAF, YY1, PU1, USF, EGR, CMYB, MAX, ELK1, AML1, MEF3, PPAR, HOX, CP2, LEF1, etc. Such sequences can be synthesized as short oligonucleotides, e.g., 25-100 base pairs; they may also be labeled with a fluorescent moiety, and aliquoted into wells of microtiter plates such that each well contains a unique or otherwise detectable sequence, or arranged on a surface in the form of an array.

The methods of the invention further comprise the use of nucleic acid binding proteins or factors, which selectively bind cis sites in nucleic acids to modulate a genetic activity of a nucleic acid or group of nucleic acids involved in gene expression. Such factors can be of diverse origins, including mammalian, yeast, fungal and plant, for example. In one embodiment, the nucleic acid binding protein is a transcription factor and comprises, for example, a DNA-binding protein that 1) binds to a cis site, and 2) is used by a cell in transcription. A transcription factor can interact covalently or non-covalently with other factors or co-regulators to form a complex that binds a cis site. The factors within such a binding complex that bind to nucleic acid, e.g., DNA, are included within the term “transcription factor”. It is also possible that some factors within a complex are transcription-associated in that they have the potential to bind to DNA, but do not contact a cis site directly; instead such factors contact one or more other transcription factors, as mentioned above, or co-regulators, for example, SRC-1 (steroid receptor coactivator 1), CBP/p300 (CREB-binding protein), ARC (activator-recruited cofactor), (Robyr et al., 2000, Mol. Endocrin., 14:329), SDP1 (Babb and Bowen, 2003, Biochem. J., 370(Pt 2):719), RanBPM (Ran-binding protein in the microtubule-organizing center) (Wang et al., 2002, J. Biol. Chem. 277:48020, 2002), and RACK-1 (receptor for activated C kinase) (Han et al., Mol. Cell, 14:420).

A nucleic acid binding factor can be a polypeptide or a polypeptide that is modified, for example, by reactions comprising phosphorylation, acetylation, or methylation or the reversal of such reactions, or the addition or removal of one or more carbohydrates, nucleotides, nucleic acids including RNA and DNA, cofactors, lipids or other chemical groups. A nucleic acid binding factor can also be a non-proteinaceous molecule, such as a lipid, carbohydrate or nucleic acid, or any combination thereof. The use of such nucleic acid binding factors in connection with the methods of the first avenue for forming complexes as described herein can comprise a diverse population of nucleic acid binding factors. As used herein, the term “diverse population of nucleic acid binding factors” means a composition containing a plurality of different nucleic acid binding factors. The greater the number of different factors within the population, the greater the diversity of the population.

A population of different nucleic acid binding proteins, regulatory proteins, and co-regulatory proteins (i.e., a diverse population of these molecules) can be of low diversity for certain applications of the method. For example, a population of nucleic acid binding proteins, regulatory proteins, or co-regulatory molecules of low diversity includes 2, 3, 4, 5, 6, 7, 8, 9, about 10 to 20, about 21 to 50, about 50 to 100, or about 50 to 500 different nucleic acid binding proteins, regulatory proteins or co-regulatory molecules. A population of nucleic acid binding proteins, regulatory proteins, or co-regulatory molecules of higher diversity includes more than about 100, more than about 10³, more than about 10⁴, more than about 10⁵, or more than 10⁶ different nucleic acid binding proteins, regulatory proteins, or co-regulatory molecules, such as are determined by proteomic studies, or 2-dimentional gels, for example. Such diversity can, for example, originate in all nucleic acid binding proteins, regulatory proteins, or co-regulatory proteins found in a cell or cellular extract. As with the diverse populations of isolated nucleic acid molecules, the members within a diverse population of nucleic acid binding proteins, regulatory proteins, or co-regulatory proteins can be known, unknown or partially known so long as at least two of the factors are different.

In one aspect of the invention, a plurality of nucleic acid binding proteins, regulatory proteins, and co-regulatory proteins comprises all of these molecules present inside a cell or cell population of interest. In another aspect of the invention, a plurality of complexes of nucleic acid molecules and nucleic acid binding proteins, regulatory proteins, or co-regulatory proteins comprises from 2-10 complexes, from 10-100 complexes, from 100-500 complexes, from 500-1000 complexes, from 10³-10⁴ complexes, or from greater than 10⁴ complexes.

The methods of the invention also comprise the use and detection of co-regulatory molecules (co-regulatory proteins or co-regulators). Co-regulators are molecules that bind to nucleic acid binding molecules or other co-regulators, and contribute to the activity or function of the other molecules or the complexes in total. For example, co-activators and co-repressors bind to transcription factors that are bound to cis sites, and thus alter the activity of the complexes in the transcription process. Alternatively, co-activators and co-repressors bind to transcription factors and/or other co-regulators that are free in the cells, and then those complexes, in turn, bind to cis sites, resulting in a change in activity of the complexes and the genes regulated by those complexes. Binding of co-regulators to transcription factors can lead to the binding of the transcription factors to cis sites, whereas, in some cases, transcription factors without co-regulators may not be able to bind to their nucleic acid binding sites.

The methods of the present invention are applicable to the profiling of gene regulatory element activity of a wide variety of nucleic acid types and sizes, and from any organism. In one embodiment involving regulatory element complex formation in accordance with the present invention, a library or plurality of nucleic acid molecules, each comprising at least one, and preferably different, cis sites, is combined or contacted with a protein-containing (and possibly nucleic acid-containing) extract from a cell population of interest under conditions that allow the formation of specific nucleic acid-protein complexes. The resulting complexes can comprise cis site-nucleic acid binding factor complexes, or cis site-nucleic acid binding factor-co-regulator complexes (also called cis site-regulatory protein complexes) under appropriate conditions. Gene regulatory elements are determined to be active as a result of their ability to form such cis site-regulatory protein complexes under appropriate cell-free conditions or inside living cells. The specific nucleic acid-protein complexes are characterized and quantified for binding activity as a measure of gene regulatory element activity in the original cell population.

The complexes formed comprise, for example, one cis site plus one nucleic acid binding factor, one cis site plus more than one nucleic acid binding factor, more than one cis site plus one nucleic acid binding factor, or more than one cis site plus more than one nucleic acid binding factor. Such complexes also comprise a combination of one or more cis sites or transcribed regions plus one or more nucleic acid binding factors plus one or more co-regulating molecules. Further, complexes comprise one or more nucleic acid binding factors plus one or more co-regulator proteins, such that the complex has the capability to bind to its appropriate cis site. Similarly, complexes also comprise co-regulator-co-regulator complexes, such that each complex has the capability to bind to an appropriate nucleic acid binding factor in the process of regulating gene expression. In one aspect involving the process of transcription, complexes comprise a combination of one or more cis sites or one or more transcribed regions, plus 1) one or more transcription factors, 2) one or more members of the pre-initiation complex or 3) one or more members of the transcription machinery.

Protein extracts containing the nucleic acid binding factors involved in the methods comprise, without limitation, nuclear extracts, cellular extracts, cytoplasmic extracts, extracts from cells used for expressing (producing) a particular biomolecule, such as a protein, mitochondrial extracts, cell membrane extracts, or chloroplast extracts. Proteins contained within the extracts can be full-length proteins, partial proteins, polypeptides or portions or fragments thereof, e.g., peptides or oligopeptides.

In the second avenue of forming regulatory complexes in accordance with the present invention, i.e., involving cell-based methods, cis site-regulatory protein complexes or transcribed region-regulatory protein complexes that form in living cells and are involved in gene regulation, or have the potential to be involved in gene regulation, are detected and analyzed. Such complexes can be analyzed while still within the cells in which they formed, e.g., in situ analyses. Alternatively, the complexes formed inside of the cell can be analyzed following isolation from the cell. The complexes can be isolated by breaking open or lysing the cells and the cell nuclei, using methods and reagents conventionally known in the art, and then isolating all nucleic acid-protein complexes, or only specific nucleic acid-protein complexes. For instance, cells or cell nuclei can be lysed using detergent solutions, such as SDS or deoxycholate, or by physical or mechanical means, such as by passage through a nozzle or a needle, or by sonication.

Components of the complexes can be cross-linked together before isolation and/or analysis to ensure stability of the complexes during isolation or other manipulations. Cross-linking can be carried out using chemicals or biological fixatives, such as formaldehyde or paraformaldehyde, or using physical means, such as ultraviolet (UV)-light. One aspect comprises reversible cross-linking, so that the proteins and nucleic acids that were once linked together can be subsequently separated from each other. Such cross-linking can utilize specific linker moieties that are cleavable in order to allow separation of the nucleic acid binding sites from their nucleic acid binding factors. Another aspect utilizes cross-linking methods that have mild or essentially no effects on the nucleic acid binding factors and co-regulators so that the molecules are more easily characterized after separation by methods such as mass spectrometry. Cells can also be treated with various compounds, for example, dIdG or other repeating dinucleotides, or exposed to certain environmental conditions, for example, heat or certain buffer conditions, to minimize nonspecific or other non-regulatory nucleic acid-protein complexes.

In the methods of this invention in which regulatory element complex formation takes place inside of cells, cis site-regulatory protein complexes or transcribed region-regulatory protein complexes are obtained or isolated from the cell population of interest, and then complexes containing specific nucleic acid binding factors or specific nucleic acids are partitioned away from the rest of the mixture. This is accomplished by use of affinity reagents that recognize and bind to particular nucleic acid binding factors or co-regulators, including polyclonal or monoclonal antibodies, portions of antibodies, preferably, binding portions, intrabodies, single chain antibodies, receptors that recognize a ligand, and the like. Portions of the nucleic acid binding factors and co-regulators that are specifically recognized include certain epitopes that comprise the molecules, epitopes that can be induced or that can change under certain conditions, or added tags or peptides to which a particular affinity reagent is generated. Alternatively, the affinity reagent recognizes an epitope found in common among a class of nucleic acid binding factors or co-regulators, thus allowing the isolation of complexes comprising a certain class of gene regulatory molecules. In another embodiment, the affinity reagent is directed against another molecule, which itself binds to the nucleic acid binding factor or co-regulator. Physical means, including molecular weight sizing or partitioning by charge, can also be used to separate certain complexes or groups of complexes.

In related embodiments, affinity reagents bind first to another molecule, which itself binds to a regulatory element such as a cis site, or a nucleic acid binding factor, or a co-regulator. Polyclonal or monoclonal antibodies can be used, as well as portions of antibodies such as fragments, e.g., Fab, Fab′, intrabodies or single chain antibodies. Antibodies, or portions thereof, can also be directed against a tag peptide or protein that is synthesized as part of the regulatory protein or is linked to the protein. Alternatively, affinity reagents that recognize a conserved epitope or other epitope shared by or in common among a class of regulatory elements are used, thereby allowing the isolation of a specific class of regulatory complexes.

In certain embodiments, the affinity reagent recognizes and binds to a general transcription factor. In other embodiments, the affinity reagent recognizes and binds to a transcription factor for which the cis binding sites are limited to a subset of genes. Antibodies that recognize co-regulating proteins such as co-activators and co-repressors, which themselves bind to certain nucleic acid binding proteins, are used in this invention. Similarly, antibodies that recognize chromatin-modifying enzymes such as histone-acetylating (or deacetylating), histone-methylating (or demethylating), or other chromatin-remodeling enzymes are used. Antibodies that recognize components of the PIC or the transcription machinery are also used.

Affinity reagents include any molecules or compounds that specifically recognize and bind to any part of a nucleic acid regulatory region or cis site, or to a regulatory protein. Affinity reagents may be used that can discriminate between regulatory proteins involved in active transcription and those not involved in transcription, e.g., if the regulatory protein undergoes some chemical modification that influences its activity. Examples of affinity reagents other than immunoreagents include receptor-ligand components, nucleic acid aptomers, nucleic acid sequences, and naturally occurring interactants, such as biotin and avidin or streptavidin.

In other embodiments involving transcriptional regulation, the affinity reagent is specific for a particular transcription factor or other regulatory protein so that all complexes containing that factor or protein can be isolated. If a particular type of cis site is present in a low number of copies in the genome being studied, the number of cis site-regulatory protein complexes isolated is likely to be low. If the particular cis site is more abundant in the genome, a larger number of cis site-regulatory protein complexes is isolated. Thus, a subset of genes regulated by a particular transcription factor can be identified based on the isolation of sequences adjacent to, or overlapping, the coding regions for these genes. In one example, nucleic acid-protein complexes from a particular cell type such as Jurkat cells are exposed to an antibody that recognizes and binds to a specific transcription factor. All complexes containing that transcription factor are immunoprecipitated together, and the nucleic acid molecules that are pulled down in this reaction are analyzed for their base sequence. Once the fragment sequences are determined, they are mapped on the appropriate genome using publicly available databases and search functions such as NCBI's (National Center for Biotechnology Information) BLAST® (Basic Local Alignment Search Tool), the University of California, Santa Cruz genomic browsers (e.g., Human Genome Browser Gateway), and Ensembl Genome Browser from the Sanger Institute, Cambridge, England. These fragments are typically located in the promoter regions upstream of genes (to the 5′ direction of genes) and generally within 1000 bp of the transcriptional start sites. Since the fragments that are pulled down have been randomly cleaved, e.g., by sonication, to lengths varying from 200-1000 bp, they will either be immediately 5′ to the first exon or will overlap the first exon of each gene.

In another embodiment, the affinity reagent is specific for a general factor, such as a general transcription factor, that can be bound at most or all sites in the genome where transcription is initiated. These factors contribute to the transcription pre-initiation complex or initiation of transcription, or modify chromatin proteins, such as histones, by means of acetylation, methylation, and/or phosphorylation. These types of factors bind directly to nucleic acid, or they bind to other molecules that are nucleic acid-binding in nature. General factors can be bound to their cis sites at all times, or can activate a process involved in gene expression only when another factor or co-regulator is present, for example, when the other factor is bound in close proximity to its cis site, or bound to the general factor itself. In certain cases, the isolation of complexes using more than one affinity reagent is used to analyze the presence of complexes containing multiple nucleic acid binding factors and/or coregulatory molecules. Thus, sites of transcription initiation are globally determined, i.e., throughout the genome, by isolating and analyzing the specific regulatory complexes that are formed involving nucleic acid binding factors known to be involved in many, or possibly all, sites where transcription starts.

In another embodiment, affinity reagents that recognize components of the transcription machinery or molecules otherwise involved in the transcription process are used to isolate complexes containing actively transcribed regions of genomic DNA. These regions comprise coding sequences of genes encoding proteins of known function, known genes of unknown function, predicted genes or open reading frames of new, previously unidentified genes. The quantification of complex formation containing regulatory elements or other molecules involved in active transcription is useful for determining the transcription rates of genes whose sequences are found in such complexes.

Methods of analysis of the isolated fragments containing cis sites include any techniques that can determine the base sequence, or a portion of the base sequence, of the fragments. An example of one such method involves direct sequencing of the fragments or portions of the fragments, using methods routinely practiced in the art and sequencing equipment as sold by any number of vendors, for example, Applied Biosystems. Another exemplary method involves sampling shorter portions of each fragment by amplification of the sequences using specific or randomly generated primers into a library. With this method, a wide variety of overlapping fragments are generated and those of a certain length, e.g., 50-100 bp in length, are selected by size fractionation methods such as electrophoresis of the entire mixture on a gel and then elution of the fragments in the desired size range. These short fragments are concatamerized into chains of 10-20 fragments and cloned into a cloning vector in order to amplify and purify each concatamer for standard sequencing. With this method, sequencing of one concatamer of approximately 20 short fragments yields information on about 20 of the longer fragments isolated as a consequence of protein binding. Because the 45-50 bp sequences are of more than adequate length to be unique in a eukaryotic genome, each of the fragments can be mapped in the appropriate genome and relationships with other annotations, such as gene positions, can be readily established.

Another illustrative analysis method involves amplifying the nucleic acid sequences using primers located on either side of sequences believed or postulated to be present in the selected regulatory complexes. PCR primers are typically about 20 nucleotides in length and are designed to flank specific internal regions using the publicly available genomic sequence databases. When the correct internal fragments are found to be amplified, either by the correct size or verification methods, such as Taqman or hybridization of probes to the internal sequences, it can be concluded that the internal sequence was present in the original regulatory complexes.

The nucleic acid fragments can also be analyzed by hybridization to other nucleic acids of known sequence, as commonly practiced in the art. In this case, the nucleic acid fragments are first denatured into separate strands if originally double-stranded, and the nucleic acids to which they can hybridize are also single-stranded. In some cases, the unknown nucleic acid fragments are amplified, e.g., by PCR, before hybridization in order to ensure that an adequate amount of each nucleic acid is available. In some cases, a detection label, e.g., a radioactive tag, an enzymatic tag, a fluorescent tag, or a chemiluminescent tag, is included to allow detection of the specific hybrids. Hybridization can take place using a wide variety of formats, e.g., in solution, such as in tubes or wells of plates; on macroarrays, such as on filters or membranes; or on microarrays comprising hundreds or thousands of the various known nucleic acid sequences attached or otherwise placed thereon. Hybrids are detected by methods known to those in the art, comprising autoradiography, fluorimetry, luminometry, and phosphoimage analysis.

Another embodiment of the global profiling methods of the present invention relates to the discovery of novel cis sites for nucleic acid binding proteins. Those nucleic acid fragments or sequences that exhibit a significant level of protein binding according to the present invention, as determined by any method of analysis (e.g., as described herein), are considered sites for sequence-specific protein binding. Isolation of the nucleic acid, or nucleic acid segments, containing the sites that specifically bind proteins and comparison of their sequence(s) with known cis site sequences are then performed to determine if these sites belong to a class of known protein binding sites or if they are novel protein binding sites, i.e., they have no recognizable homology or only partial homology, for example, half of the site is homologous to known protein binding sites.

Whether they contain known or unknown cis sites, the nucleic acid molecules useful for global profiling can be used or detected in assays, either in solution or on a solid surface. With respect to use of known cis sites on a surface, individual nucleic acids containing specific cis sites can be applied to the surface, preferably, in an organized array, so that specific cis sites have a known position. With respect to nucleic acids containing other sequences and sequences generated from genomic sources, such sequences can be individually cloned, followed by specific placement on an array, or cloned in a group and then layered onto a surface, e.g., in a known or unknown pattern. When layered onto a surface, individual molecules can be globally assayed by any number of methods. For example, antibodies can be generated to either specific nucleic acid sequences, or to specific proteins, using methods known in the art for generating nucleic acid-specific, or protein-specific antibodies (Stollar, 1986, CRC Crit. Rev. Biochem., 20:1; Milgrom, 1985, Pharmacol. Ther. 28:389). Following the formation of nucleic acid-protein complexes, the antibodies are employed to screen for the complexes. Specific methods for carrying out such screening are well understood by those of skill in the art, e.g., preblocking with protein mixtures to prevent nonspecific binding of the antibodies, contacting the antibodies with the complexes to allow them to bind to their specific epitopes, washing away of unbound antibodies, and then detection of the antibodies. Antibody detection is accomplished by use of secondary antibodies that bind to the first (primary) antibodies, or by detecting tags that are attached to the primary or secondary antibodies. Tags include enzymes for which the substrate can be added (Voller et al., 1978, J. Clin. Pathol., 31:507), or compounds such as biotin for which avidin or streptavidin is used for detection (Diamandis and Christopoulos, 1991, Clin. Chem., 37:625).

When cis site-containing nucleic acids are used in global profiling in solution, detection of complex formation involving regulatory proteins is also achieved by the use of an array of molecules that can detect one particular component or a class of components involved in the complexes. For example, high affinity polyclonal or monoclonal antibodies, raised against either nucleic acid binding proteins, or portions of the nucleic acids containing the cis site involved in binding, comprise the array. Preferably, the proteins or nucleic acids are of known composition or identity. Further, such antibodies can be placed or arrayed on a solid support in a manner analogous to the cis site arrays. The mixtures of complexes from cells or from cell-free binding reactions are then contacted with the antibody-containing array. Binding of the cis site-regulatory protein complexes to the antibodies is then detected by any suitable technique, for example, by using various labeled probes, e.g., a probe that binds specifically to the nucleic acid or a different probe, such as another antibody or group of antibodies, that binds specifically to the protein.

Nucleic acid molecules, as described above, can be mixed with a population of cellular proteins in solution under conditions that promote sequence-specific nucleic acid-protein interactions and the level of protein binding to each individual nucleic acid molecule can be measured directly by an appropriate detection method such as fluorescence polarization. Binding of several known nucleic acid binding factors to their cis sites can be monitored simultaneously by fluorescent detection of two or even three distinguishable fluorescent tags. A known nucleic acid sequence comprising a known binding site, along with its corresponding binding factor, can be used as an internal control for validating binding conditions and for quantifying the level of protein binding to the nucleic acid, e.g., DNA molecules (unknowns).

Another embodiment of the present invention encompasses comparing the global gene regulatory activity profiles for two different cell populations and determining which elements exhibit differential activity between the two populations. Such methods comprise comparing the quantity of active cis site-regulatory protein complexes or transcribed region-regulatory protein complexes that are formed in one cell population with the active complexes that are formed in the other cell population. Cell populations that can be compared include, for example, and without limitation, different cell types within the same organism, the same cell type between or among different organisms, normal versus diseased cells of the same types, normal versus transformed cells of the same types, cells at different stages of differentiation or development, cells treated with an exogenous material such as a drug compound or other therapeutic molecule versus untreated cells, cells exposed to two different compounds or molecules, cells exposed to a different external or internal condition versus unexposed cells, cells exposed to two different external or internal conditions, or cells within a comparison comprised of more than two different cell populations. In this aspect of the invention, regulatory element activity profiles obtained for the different cell populations are directly compared in order to determine differences in gene regulatory activity. Accordingly, gene expression is thus directly compared between the two (or more) populations. In a further related aspect, profiles obtained from cells at different metabolic or physiologic states are compared (preferably using cells from the same source, or closely related sources) in order to determine differences in gene regulatory activity and gene expression.

In accordance with this invention, the cells to be tested for gene regulatory element activity can be in any state of metabolism or under any physiologic condition. For example, in one aspect, cells are treated with one or more compounds that affect the cells' metabolic or physiologic status. Such compounds are administered at one or more concentrations, as determined from various assays that test for particular effects, for example, the ability to induce changes in cell behavior, viability, differentiation, and so on, or from data obtained from other cell types, or from data obtained from similar compounds, and the like. The cells can also be pre-treated with other molecules prior to adding the particular compound of interest and then compared with cells not pre-treated. Alternatively, other compounds can be added after the cells are exposed to the first compound(s), and/or environmental conditions under which the cells are grown can be changed. Following the addition of such compounds and/or alteration in environmental conditions, the cells of interest are globally profiled for changes in their gene regulatory element activity.

In some embodiments, the present invention allows the assay of nuclear extracts containing nuclear proteins, for example, activators, repressors, transcription factors, proteins involved in RNA function (for example, splicing, trafficking, degradation) or chromatin structure formation, maintenance, and/or remodeling, that are obtained from cells of interest either before or after exposure to compounds or environmental conditions in the form of extracts or complexes. In other embodiments, proteins comprising cytoplasmic proteins and membrane-bound proteins are obtained from cells of interest using methods conventionally practiced in the art and profiled according to the instant invention. In still other embodiments, cellular extracts comprise cis site-regulatory protein complexes or transcribed region-regulatory protein complexes. In any embodiment, such extracts or complexes can be obtained at a single time point following any change to the cells such as exposure to a compound, or at different time points over a short or long period of time.

Cells amenable to global profiling for regulatory element activity and from which protein extracts containing regulatory elements or nucleic acid-regulatory protein complexes can be obtained include animal (e.g., mammalian, vertebrate, invertebrate) cells, plant cells, fungal cells, Archaea cells, insect cells, protozoans, algal cells, yeast and bacteria. Animal cells can include, without limitation, avian, bovine, canine, equine, feline, fish, human, rodent (both murine and rat), ovine, porcine, and primate cells.

In addition, cells can comprise cell-like structures, including cells infected with pathogens such as viruses, prions, bacteria, fungi, yeast, parasites, other microorganisms, and portions thereof. The cells can be obtained, without limitation, from in vivo or in vitro (including ex vivo) sources, including tissues, organs, or whole organisms, e.g., via biopsy, cell sloughing, in a blood sample, or via a body fluid or specimen, such as saliva, sputum, stool, cerebrospinal fluid (CSF), urine, and the like. Such cells can be normal, diseased, transformed, infected with a virus, pathogen or other exogenous organism, transfected or transformed with an exogenous gene, portion of a genome or genome, treated so as to represent a particular state of typical or a typical growth or maintenance, or represent a particular stage of development. Nonlimiting examples of cell types embraced by this invention include fibroblasts, epithelial, endothelial, hematopoietic, CNS-derived, bone-derived, myocytes, stromal cells, stem cells, basal cells, germ line cells, blood cells, cells from organs, e.g., cervical, ovarian, prostate, testes, liver, lung, kidney, pancreas, stomach, intestine, esophagus, brain, heart, and the like.

In certain embodiments, the methods of the invention employ assay formats that use diverse populations of nucleic acid molecules comprising one or more cis sites, diverse populations of nucleic acid molecules with the potential for being transcribed, diverse populations of nucleic acid binding factors, and diverse populations of co-regulators. In a further embodiment, such elements are used in an array format such that different nucleic acid molecules containing different cis sites, different transcribed regions, different nucleic acid binding factors, or different co-regulators are positioned at separate locations on the array.

In embodiments of the invention, testing for regulatory complex formation can be carried out by determining changes in the polarization of a fluorescent reference tag using fluorescence polarization over a predetermined time period (Hill and Royer, 1997, Meth. Enzymol., 278:390). This technique provides direct, nearly instantaneous measurement of a labeled molecule's (i.e., tracer's) bound/free ratio, even in the presence of free tracer. Fluorescence polarization is a measure of the time-averaged rotational motion of fluorescent molecules. A fluorescent molecule, when excited by polarized light, will emit fluorescence with its polarization primarily determined by the rotational motion of the molecule. Since molecular rotation is inversely proportional to the molecular volume, the polarization is, in turn, related to the molecular size. A small molecule rotates fast in solution and exhibits a low value of polarization, while a large molecule exhibits a higher polarization because of its slower motion under the same conditions. Thus, changes in fluorescence polarization reflect the association or dissociation between molecules of interest, including nucleic acid binding factors and nucleic acid, e.g., DNA or RNA, fragments comprising their cognate binding sites.

In yet another embodiment, known nucleic acid molecules on an array are contacted with the cis site-containing nucleic acid molecules isolated as a result of being bound with at least one nucleic acid binding factor. The nucleic acid molecules in the isolated mix that hybridize to the nucleic acid molecules on the array are therefore identified. Hybridization is carried out under conditions corresponding to moderate stringency followed by washing away unhybridized molecules using conditions corresponding to high stringency. Stringency conditions determine the amount of mismatch between the nucleic acid strands that form duplexes, where high stringency conditions involve detection of identical or very highly related sequences (up to 5% mismatch), and moderate stringency conditions allow hybrids containing 10-20% mismatched hybrids. Stringency is generally determined by the salt concentration and the temperature. As a guide, high stringency conditions involve a salt concentration of 0.1×SSC and a temperature of 68° C., for example; moderate stringency conditions involve a salt concentration of 0.2-0.5×SSC and a temperature of 42° C., for example; and low stringency conditions involve a salt concentration of 2×SSC at room temperature (e.g., 25-35° C.), for example, where SSC typically comprises 0.15 M Na citrate, 1.5 M NaCl.

In another embodiment, the specific nucleic acid-regulatory protein complexes are detected and identified by the following methods or protocols: 1) direct sequencing of the bound nucleic acid molecules and analysis in silico (by computer software) for cis sites or transcribed regions within the nucleic acid sequences using known cis site motif databases (for example, Transfac by Biobase (Braunschweig, Germany) and/or genomic databases (Human Genome Browser and Mouse Genome Browser from the University of California, Santa Cruz, Ensembl Genome Browser from the Sanger Institute, Cambridge, England, and GenBank at NCBI (National Center for Biotechnology Information); or conversion of the bound RNA molecules to DNA by reverse transcription, followed by direct sequencing of the resulting cDNA and analysis for cis sites or gene regions; 2) other methods that detect at least one of the components in the complex, i.e., the nucleic acid molecule or the regulatory protein, in a bound state, such as a homogeneous luminescent assay (e.g., the Amplified Luminescent Proximity Homogeneous Assay or AlphaScreen from Perkin Elmer) where a homogeneous assay allows the detection of specific interactions without the need for separating away the unreacted components; 3) biochemical or physical characterization of the bound nucleic acid binding factors and co-regulators; 4) hybridization to the bound nucleic acid molecules using specific labeled nucleic acid probes containing cis sites or gene regions, e.g., acridinium ester-labeled probes in a homogeneous assay format; 5) separation methods based on molecular size, such as capillary electrophoresis; 6) separation methods based on other physical properties, e.g., charge, the presence or orientation of specific moieties, and secondary structure; and 7) detection using antibodies directed against proteins associated with gene expression regulation or various chromatin structures.

In a preferred embodiment, the methods of this invention are performed in a cell-free state, preferably in a moderate to high throughput format, in which more than about 10, preferably more than about 100, 1,000, or 10,000 elements can be profiled at one time. The format can include an array, in which either specific nucleic acid molecules, or combinations thereof, are located in specific locations, such as on microtiter plates, beads, slides, gels, columns, membranes (e.g., nylon, nitrocellulose, teflon, and the like), microarrays, tubes, chips, and the like. Alternatively, the format can include an array where either nucleic acid binding factors or combinations thereof are located in specific locations, such as on microtiter plates, beads, slides, gels, columns, membranes (e.g., nylon, nitrocellulose, teflon, and the like), microarrays, tubes, chips, and the like.

Within each plurality of regulatory element components or molecules that detect regulatory elements, individual nucleic acid molecules, proteins or detection molecules can be located in separate and distinct locations. The format can also include arrays or other solid supports containing detection molecules for nucleic acid-regulatory protein complexes, such as antibodies that bind to proteins associated with transcription or chromatin structures, or nucleic acid molecules that bind specifically to cis sites.

In another embodiment, methods of this invention are provided in which the complexes are formed and/or detected in solution (e.g., standard buffer conditions or with additives such as dinucleotide polymers to decrease nonspecific binding), on solid surfaces (e.g., filters, glass slides, nylon membranes), on solid supports (e.g., on microarrays, chips, or on beads), in semi-solid medium, in gels, in column matrices, in polymer formulations (e.g., in the presence of space-filling materials such as dextran sulfate, in aqueous formulations, in organic solutions, or in inorganic solutions. In yet another embodiment of the invention, the complexes are formed inside living cells, and then isolated and further analyzed in solution, on solid surfaces, on solid supports, in semi-solid media, in gels, in column matrices, in polymer formulations, in aqueous formulations, in organic solutions, or in inorganic solutions.

In other embodiments, the detection of gene regulatory element activity comprises the detection of changes in the condition(s) of one or more labels either attached to the cis site-containing or transcribed region-containing nucleic acid molecules (including plasmids), or incorporated into proteins that can bind such elements. For example, a radioactively labeled amino acid or nucleotide is used. Radioactively labeled nucleotides are incorporated into nucleic acids by use of enzymes such as polymerase, thermostable polymerase, terminal transferase, reverse transcriptase, and polynucleotide transferase, or by de novo synthesis. Radioactively labeled amino acids are incorporated into proteins during the synthesis process, either by biochemical synthesis using synthesizing instruments, by incorporation in cell-free reactions, or by incorporation in vivo in prokaryotic organisms or in eukaryotic cells. Other labels comprise, for example, chemiluminescent tags, fluorescent tags or specific enzymes. In addition, changes in fluorescence can be determined such as by fluorescent polarization. Other detection methods will be apparent to those skilled in the art upon reading this specification.

In a particular embodiment, extracts of cells of interest for testing are prepared and applied to the cis site-containing nucleic acid molecules on an array. The nucleic acid molecules on the arrays are then examined for binding of nucleic acid binding factors to the cis sites. It is to be understood that it is not necessary to remove cellular extract material containing unbound proteins prior to detecting the presence of proteins bound to the cis sites. In embodiments in which the cis site-containing nucleic acids are in solution, it is frequently useful to separate bound complexes, e.g., cis sites bound to regulatory proteins, from the unbound matter using techniques known in the art (although it is not necessary to remove unbound proteins of the extract).

Depending on the particular assay, for example, an assay in which the nucleic acids undergoing cis site analysis are in solution rather than positioned on a fixed array, complexes formed by the binding of the cis sites with nucleic acid binding proteins are separated from unreacted portions of the extract/library/mixture. For example, when the nucleic acids are in solution, complexes are isolated simultaneously as a group for further processing and detection of individual cis site-regulatory protein complexes. In contrast, when the nucleic acids are bound to a solid support, e.g., as a nucleic acid library in the form of an array, labeled proteins that interact therewith can be detected directly. Those in the art will appreciate that an unlabeled nucleic acid binding factor bound to its cognate cis regulatory site can be detected in other ways, for example, using detectable antibodies or other epitope-specific affinity reagents.

In further embodiments, profiling results of assays are compared with results from one or more control assays. In certain preferred embodiments, a control assay involves obtaining a protein extract, cis site-regulatory protein complexes, or transcribed region-regulatory protein complexes from cells that have not been exposed to compounds or changes in environmental conditions, or that have been exposed to compounds under different conditions, for example, at different concentrations, or for differing periods of time, and so on.

In still further embodiments, differences in the expression of nucleic acid binding factors, as indicated by the differences in the makeup of cis site-regulatory protein complexes, provide data valuable for determining gene regulatory element activity. Moreover, such data are provided by the methods of the invention at a global level for any cell or cell population tested. Thus, not just specific and/or known regulatory elements are tested for activity, but many, and potentially all, regulatory element activities are detectable. Global regulatory element activity profiles can be made up of regulatory element activity information obtained by use of any or all methods of the present invention, derivatives thereof or a combination of these methods. These methods may be performed simultaneously or in series to obtain information about the activities of regulatory elements. Data obtained from other methods about regulatory element activity may also be included, such as RNA profiling data involving RNAs that encode regulatory elements. Thus microarray hybridization data using labeled cDNA (complementary to mRNA), by so-called “RNA profiling”, that detects and quantifies RNA encoding transcription factors, other nucleic acid binding proteins, and co-regulators may be added to global regulatory element profiles.

Notwithstanding the complexity of the results presented by such global profiling, the methods of the invention allow for deciphering data so retrieved. For example, in any one global profile, many regulatory elements can be involved, such as those elements that regulate the expression of more than one gene, or numerous elements that regulate different genes. In some embodiments where nucleic acid molecules are presented in an array, particular regulatory elements are identified directly and the genes with which the regulatory elements are functionally associated are also directly determined. By “functionally associated” is meant those genes over which the element has some regulatory influence, be it activation, repression, sequestering in chromatin, etc.

In other examples, such as those in which libraries of regulatory elements are assayed to reveal new cis site sequences, databases listing nucleic acid binding factors that bind thereto are queried to determine which genes the cis sites are proximal to in the genome. As is understood by the skilled practitioner, such databases include lists of genes whose expression is at least partially controlled by the cis site of interest. Applicable databases include the Eukaryotic Promoter Database (Swiss Institute for Experimental Cancer Research), Transfac by Biobase (Braunschweig, Germany), and NCBI (National Center for Biotechnology Information, Bethesda, Md.). From such information, some or all of the genes whose expression is influenced by a particular regulatory element are identified. Accordingly, a nucleic acid array containing hybridization probes specific for some or all of the genes functionally associated with the particular regulatory element (or set of particular regulatory elements) is prepared. Carried to its conclusion, a database of all regulatory elements and the genes whose expression they control can be developed.

### EXAMPLES

The laboratory procedures and protocols in cell culture, chemistry, microbiology, molecular biology and cell science used below are typically well understood and commonly employed in the art. Conventional methods are used for these procedures, such as are found and provided in the art and in various general references, e.g., Sambrook et al., Molecular Cloning: A Laboratory Manual, 2^{nd} Edition, Cold Spring Harbor Press, Cold Spring Harbor, N.Y. (1989), and thus are not further described in detail herein.

### Example 1

### Growth and Treatment of Cells

Jurkat cells (human T cell line; ATCC Number TIB-152) were grown in RPMI 1640 medium supplemented with 10% fetal bovine serum, antibiotics/antimyotics, 1% L-glutamine, and 1% non-essential amino acids. At a cell density of 1-5×10⁶ cells/ml, an equal number of cells were treated with either 100 ng/ml Phorbol 12-myristate 13-acetate (PMA) plus 2 μg/ml Ionomycin in DMSO (activated Jurkat), or DMSO alone (resting Jurkat), both for 2-3 hours. Cells were washed with cold (4 C) phosphate-buffered saline (PBS) and then used for profiling.

Rat pheochromocytoma (PC) cells (cell line PC12; ATCC Number CRL-1721) were grown in high glucose Dulbecco's Modified Eagle Medium (DMEM) in the presence of 10% horse serum, and then transferred to serum-free DMEM plus N2 supplement (Invitrogen) containing 200 ng/ml Nerve Growth Factor-beta (NGF-beta; Sigma). After a 5 hour exposure, the cells were removed from the dishes, washed with PBS, and collected by centrifugation for regulatory element activity profiling.

MCF7 cells (human breast cancer cell line; ATCC Number HTB-22) were grown in DMEM supplemented with 10% fetal bovine serum, antibiotics and 2 mM L-glutamine. At 50% confluence, cells were treated with tamoxifen (5 μM), Taxol (10 nM), or doxorubicin (1 μM) for 6 hours (all drugs from Sigma). Control cells were mock-treated with 0.1% ethanol, the solvent for the drugs. Cells were washed with PBS, and nuclear extracts were prepared as described below (Example 2.A.1.).

### Example 2

In this example, global profiling was carried out as a diagnostic tool for detecting activated T cells as an indication of inflammation.

A. Formation of Regulatory Complexes Using Cell-Free Binding

1. Nuclear Extracts

Nuclear extracts were prepared according to standard methods (e.g., Dignam et al, 1983; Nucleic Acids Res. 11:1475) by hypotonic lysis in 10 mM Hepes, pH 7.9, 1.5 mM KCl, 0.15% NP-40 containing protease inhibitors on ice, and then pelleting of nuclei by centrifugation and extraction of proteins in Hepes buffer containing 420 mM NaCl. Extracts were dialyzed or diluted to 100 mM NaCl, normalized to the same protein concentration, and stored at −80° C.

2. DNA Library Preparation

A genomic DNA library containing fragments representative of human genomic DNA was generated by a method similar to that described by Singer et al. (1997, Nucleic Acids Res. 25:781-786). A mixture of primers, each containing a fixed 5′-region (18-22 bp in length) and a 9-nucleotide randomized extension at its 3′-end, was annealed to denatured genomic DNA and extended with Kienow DNA polymerase (New England Biolabs). Extension products were isolated and the process was repeated with a second mixture of primer having a different fixed region. DNA was purified and further amplified by PCR using primers containing only the fixed sequences. Amplified DNA was size-fractionated using polyacrylamide gel electrophoresis, amplified again with the same fixed sequence primers, and gel-purified to yield genomic libraries containing inserts of defined size ranges. The genomic library prepared for these studies contained inserts of genomic DNA sequences in the range of 40-45 bp in length.

3. Cell-Free Binding Reaction Using a DNA Fragment Library

For each reaction, nuclear extract was combined with library DNA, and typically included 5-10 μg of nuclear extract proteins, 5-50 ng of double-stranded library DNA, and non-specific competitor nucleic acids such as polydI:dC, salmon sperm DNA, calf thymus DNA, or E. coli total RNA. One strand of the library DNA was biotinylated at its 5′-end, so that purification from the binding reactions could be carried out using solid phase chemistry. Reaction conditions also included 1-5 mM MgCl₂, 50-100 mM KCl, 20-25 mM HEPES-NaOH, 10-20% glycerol and 0.1 mM EDTA. Reactions were incubated at 4 C for 2 hours or at 25 C for 30 minutes.

DNA-protein complexes were partitioned away from unbound components using the electrophoretic mobility shift assay (EMSA) (Garner and Revzin, 1981; Nucleic Acids Res., 9:3047-60). Complexes were eluted from the 5% polyacrylamide gel and were captured on streptavidin-coated magnetic beads. The non-biotinylated strands of the DNA fragments, representing the “protein bound” fraction of the original library, were then recovered from the beads by alkaline denaturation in 0.2 N NaOH followed by ethanol precipitation. This single-stranded DNA was amplified by PCR to a moderate level and then used in a binding reaction identical to the first reaction. The process was carried out for one more round, for a total of three rounds, and the resulting DNA fragments were then analyzed for cis sites.

4. Sequence Analysis of Protein-Bound Fragments for Cis Sites

The individual DNA fragments selected in each binding reaction were concatamerized end-to-end in chains of 10-20 fragments/chain and then cloned in the CloneAmp vector pAMP10 (Invitrogen). Of the thousands of recombinant clones generated, a representative number (500-2000 fragments) were sequenced. Sequences were analyzed for known cis sites using the software MatInspector Professional (Genomatix) and their occurrences quantified (expressed as a percentage of the total fragments analyzed). The degree to which any given cis site was observed was a measure of relative binding activity within the particular cell population, and the compilation of binding activities for that cell population comprised a global profile.

Specifically, global profiles from resting Jurkat cells and from PMA/ionomycin-activated Jurkat cells (Example 1) were obtained. As shown by the representative partial profiles (i.e., a subset of profile data) in Table 1, certain binding sites were constitutive (similar levels between the two cell populations), while others were significantly differential in their levels of activity between the two cell populations. For example, the binding sites for transcription factor complexes CREB, E47, Mycmax, NFAT, WT1 and XBP1 each showed a significant increase in binding activity, all of which have been associated with T cell activation. Activation of T cells is a hallmark of certain immune disorders, including inflammation, allergy, autoimmune diseases, tissue rejection and HIV-related diseases. The reduced binding activity of ARARNT, on the other hand, had not been reported previously.

TABLE 1

| | Resting | Activated |
|---|---|---|
| | Jurkat Cells | Jurkat Cells |
| | % of total | % of total |
| | fragments | fragments |
| ARARNT | 4.1 | 1.5 |
| AP1 C | 0.7 | 1.5 |
| ATF | 0.0 | 0.8 |
| CREB | 0.7 | 3.1 |
| CAAT | 6.8 | 5.4 |
| CETS1P54 | 2.7 | 2.3 |
| CMYB | 3.4 | 3.1 |
| E47 | 0.0 | 2.3 |
| MYCMAX | 1.4 | 3.8 |
| MZF1 | 12.8 | 10.0 |
| NFAT | 2.0 | 6.2 |
| NFY | 7.4 | 8.5 |
| Sp1 | 4.7 | 6.9 |
| USF | 6.8 | 5.4 |
| WT1 | 1.4 | 7.7 |
| XBP1 | 1.4 | 3.1 |

Another cis site-transcription factor complex found to exhibit differential binding activity between resting and activated Jurkat cells was NF-kB (binding activity via global profiling was carried out, but not shown here). Higher levels of nuclear NF-kB have been found in activated T cells relative to resting cells and associated with T cell diseases such as those listed above. Furthermore, NF-kB has been shown to regulate genes important in T cell activation, such as numerous genes coding for cytokines.

5. EMSA Confirmation of Sequencing Analysis

Confirmation of the increased binding activity in activated T cells was demonstrated by electrophoretic mobility shift assay (EMSA). Nuclear extracts obtained from both resting Jurkat cells and PMA/ionomycin-activated Jurkat cells were added to separate binding reactions. Each reaction also contained a ³²P-labeled double-stranded oligonucleotide comprising the binding site for NF-kB. Some reactions also contained competitor oligonucleotides.

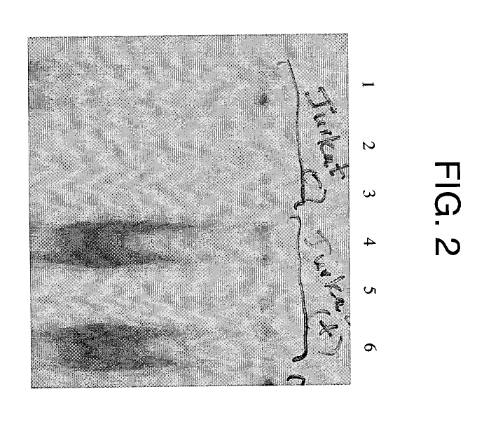

As shown in FIG. 2 (lanes 1-3), no labeled oligonucleotide shifted in the lanes from binding reactions containing resting Jurkat cell extract. As expected, no differences were seen between the lanes containing no competitor oligo (lane 1), a competitor oligonucleotide specific for the NF-kB binding site (lane 2), and a competitor oligonucleotide mismatched to the NF-kB binding site (lane 3). In contrast, a significant amount of gel-shifted material (DNA-protein complexes) was observed in lanes 4 and 6, which came from binding reactions containing nuclear extract from activated Jurkat cells. Lane 4 contained no competitor oligonucleotide and lane 6 contained mismatched competitor oligonucleotide. Lane 5, which contained matched competitor oligonucleotide, also showed no gel-shifted material, demonstrating specificity of the shifted DNA-protein complexes.

6. Cell-Free Binding Reaction Using Labeled Fragments as Binding Site DNA

Binding reactions were carried out using a defined population of short (25 bp) double-stranded oligonucleotides containing consensus binding sites for eight known transcription factors. The various fragments, differing only in their centrally located cis sites, were shown not to cross-hybridize with each other. Fragments were labeled with ³²P and used in binding reactions using the same Jurkat nuclear extracts and the same conditions as described above. Separation of DNA-protein complexes was accomplished by EMSA using a preparative 5% polyacrylamide gel, which was electrophoresed until unbound DNA fragments ran off the gel. The area containing the complexes was excised and the DNA was eluted using 0.5 M NaCl and 0.1% SDS to dissociate the DNA-protein complexes. Eluted DNA was concentrated by ethanol precipitation, redissolved in H₂O, and heat-denatured in preparation for hybridization to DNA on an array, e.g., a macroarray or microarray.

7. Analysis of Protein-Bound Fragments by Hybridization to DNA on a Macroarray

DNA filters as macroarrays were prepared by UV-crosslinking single-stranded oligonucleotides of known sequences to an Immobilon Ny+nylon membrane (Millipore). In this example, the same eight oligonucleotides containing specific cis sites used in the cell-free binding were spotted in duplicate on the arrays. Hybridization of the labeled DNA fragments, selected as a result of being protein-bound in the step prior and denatured to form single strands, was carried out using standard hybridization conditions (for moderate stringency) including 50% formamide, 5×SSPE, 1% SDS and SXDenhardt's solution at 42 C (where 5×SSPE=0.75 M NaCl, 50 mM NaH2PO4, 5 mM EDTA and SXDenhardt's solution, and the latter solution=0.1% Ficoll 400, 0.1% polyvinylpyrrolidone, 0.1% bovine serum albumin). Filters were washed under typical high-stringency conditions (0.1% SDS, 0.3×SSC, 65 C, where 0.3×SSC=0.045 M sodium citrate, 0.45 M NaCl) and then exposed directly to X-ray film for autoradiography. Radioactivity in each lane was quantified with image analysis software, and the levels of individual binding activities were determined by comparing each spot to its control spot on another filter, where the DNA fragment mixture was hybridized directly (without going through the binding reaction) and assumed to be 100% complete.

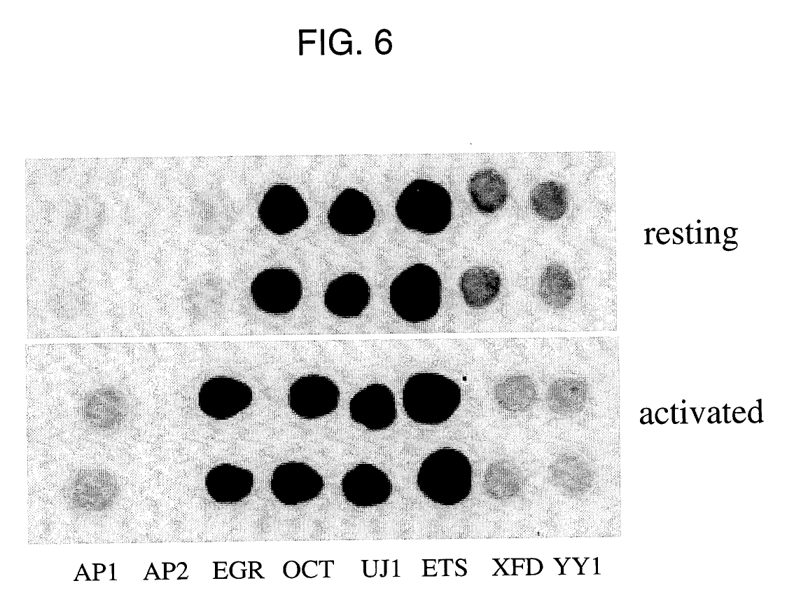

As shown in FIG. 6, a profile of activity for these eight regulatory element complexes was obtained for both of the Jurkat cell populations. Upon comparison of the two profiles, it was apparent that both AP 1 and EGR (early growth response) activities were induced in the activated Jurkat cells. These differential activities are consistent with T cell activation. Also, AP2 activity was not observed, but a novel possibly Jurkat-specific transcription binding activity named UJ 1 was constitutively present.

8. Cell-Free Binding Reaction Using DNA Fragments in Array Format

This example describes an array method of global profiling of regulatory element binding activity present in a cellular extract.

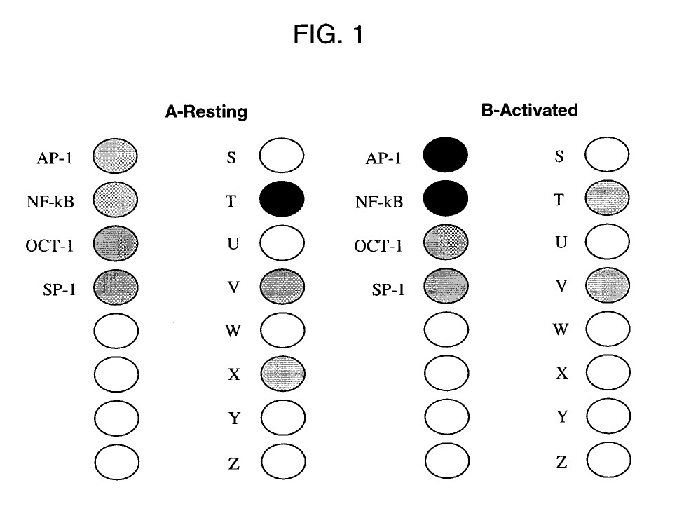

Cis site-containing molecules are labeled with a fluorescent tag and then placed on an array at a density of one protein binding site per molecule and one cis site sequence per location on the array (see, e.g., FIG. 1). Such arrays are reacted with solutions containing populations of cellular proteins under conditions that promote sequence-specific DNA-protein interactions. The level of protein binding to each type of DNA molecule is measured by fluorescence polarization (or a similar method) to quantify DNA-protein binding. The exact level of binding to each individual type of cis site by proteins contained in each cellular protein population is quantified and compared. This comparison provides a profile of differing binding activities that are present in the cells used to prepare the protein populations.

For example, the cis site specific for AP-1 protein binding can be present on two separate but identical arrays. Nuclear protein extracts prepared from both resting (DMSO-treated) Jurkat cells or PMA/ionomycin (in DMSO)-treated Jurkat cells are added to these two arrays such that proteins from resting cells are placed on one array with the AP-1 cis site nucleic acid molecule, and the proteins from PMA/ionomycin-treated Jurkat cells are placed on the other. The level of protein binding to the AP-1 site in each of the two extracts is then measured by fluorescence polarization. If binding occurs, the level of bound cis site is seen to be significantly higher following addition of the extract and therefore results in higher measurements of fluorescent anisotropy than prior to extract addition. The precise level of AP-1 binding from both protein samples and thus the level of induction of AP-1 binding by PMA/ionomycin-treatment are measured. This approach, when performed in parallel with hundreds, or even thousands, of different protein binding sites, successfully profiles the binding activities of some, many or all known and even unknown nucleic acid binding factors within different cell types. Differences in binding activities are indicative of global changes in patterns of gene expression.

As an example of such profiling, FIG. 1 shows labeled DNA molecules from the library placed into individual wells of microtiter plates such that each well contains a unique sequence that is unknown (represented by letters S-Z), or one that is known to bind sequence-specific DNA-binding proteins (for example AP-1, NF-κB, OCT-1 or SP-1). The solution can also contain nonspecific “carrier” DNA and/or internal control DNA. Identical replicates (shown as plates A and B) of the microtiter plates are preferably generated in order to profile each protein population (e.g., cellular extract) to be compared. For example, shown in FIG. 1 is comparison of resting (A) and PMA/ionomycin-activated (B) Jurkat cells.

Nuclear extracts containing populations of DNA-binding proteins are added to arrays of DNA molecules comprising binding sites for known nucleic acid binding factors under conditions that promote DNA-protein binding. Protein binding to each type of DNA molecule is monitored by changes in fluorescence anisotropy values for labeled DNA fragments over time. Those fragments that show an increase in fluorescence anisotropy values over time are scored as positives for protein binding. The greater and more rapid the increase, the lower the Kd for the DNA-protein complex. Thus, since the Kd is inversely proportional to the protein binding activity, which itself is dependent on both protein concentration and affinity of the protein for its DNA binding site, the level of binding activity for each type of complex from each protein population is thereby quantified.

In this example, if nuclear extract from resting Jurkat cells is added to plate A and nuclear extract from PMA/ionomycin-activated Jurkat cells is added to plate B, a significant increase in the fluorescence anisotropy from the AP-1 and NF-κB cis site-containing DNA molecules in plate B is expected compared to plate A, as a result of known induction of both AP-1 and NF-κB binding activities upon Jurkat cell activation with TPA/ionomycin. In contrast, a rapid and significant increase in the fluorescence anisotropy of the labeled OCT-1 and SP-1 fragments is expected in both plates A and B equally, as a result of the moderately high constitutive levels of OCT-1 and SP-1 binding activities that are found in both resting and activated Jurkat cells. Of importance, whether the binding activities are differentially active or are constitutive between two cell populations, the global profiling methods described in accordance with this invention allow quantification of the levels of binding activities. Simultaneous binding of proteins to cis sites within the same array location can also be monitored by simultaneous fluorescent detection of two or more discernable fluorescent tags.

These results illustrate the feasibility as well as the usefulness of determining global gene regulatory element activity profiles involving quantitative levels of cis site-nucleic acid binding factor activities within cell populations. These profiles are then compared between and among different cell populations to discern differences in gene expression that are important in overall genetic and phenotypic changes. The identification of such changes, as determined by the global profiling methods of the present invention, is useful in many applications in medicine, such as determining the effects of compounds on gene regulation, and recognition of disease states in cells. FIG. 1 provides an exemplary schematic of the profile determining process of this invention.

### Example 3

A. Isolation and Characterization of Regulatory Complexes from Living Cells

1. Immunoprecipitation of Regulatory Complexes

Living cells (10⁷-10⁹) were fixed with 1% formaldehyde for 10-30 minutes, after which fixation was stopped with 0.125 M glycine. Fixed cells were washed in PBS and lysed in 10 ml cell lysis buffer (5 mM PIPES, pH 8, 85 mM KCl, 0.5% NP40 and protease inhibitors) by homogenization in a dounce homogenizer. Nuclei were collected by centrifugation and resuspended in 4 ml nuclei lysis buffer (50 mM Tris-HCl, pH 8.1, 10 mM EDTA, 1% SDS and protease inhibitors). Lysate was sonicated to shear the DNA to lengths between about 200 and 1000 bp. Insoluble material was removed by centrifugation, and the supernatant containing cross-linked chromatin was snap-frozen in dry ice/EtOH, and stored.

Cross-linked chromatin was added to buffer containing 0.1% SDS, 0.1% Triton X-100, 150 mM NaCl, 1 mM EDTA and 15 mM Tris-HCl, pH 8.0 and protein A-agarose beads (previously blocked with BSA and sonicated salmon sperm DNA) were added, and reactions were incubated at 4° C. for 1-3 hr. Agarose beads were removed by centrifugation, and antibodies against general transcription factors TFIIEβ, TFIIB, TBP, or CBP, acetylated histone H3, or RNA polymerase II (1-5 μg/reaction) (obtained from Santa Cruz Biotechnology or Upstate Biotechnology) were added to the appropriate samples. “No antibody controls” were processed in parallel.

After incubation overnight at 4° C., protein A-bound agarose beads were again added to bind the antibody-antigen complexes, and the beads were washed 2 times each with low salt buffer (containing 0.1% SDS, 1% Triton X-100, 2 mM EDTA, 20 mM Tris-Cl, pH 8.1, 150 mM NaCl), high salt buffer (containing 0.1% SDS, 1% Triton X-100, 2 mM EDTA, 20 mM Tris Cl, pH 8.1, 500 mM NaCl), LiCl buffer (containing 10 mM Tris-Cl, pH 8, 250 mM LiCl, 1% Igepal CA630 (Sigma), 1% deoxycholic acid, 1 mM EDTA) and TE buffer, pH 8.0. Chromatin was eluted from the agarose beads in nuclei lysis buffer at 37° C. for 10 minutes. Eluate was RNAse-treated for 20 minutes at 37° C., followed by proteinase K digestion for 3 hours at 37° C. Remaining cross-links were reversed by heating at 65° C. for 4 hours, and the DNA was phenol-extracted and ethanol-precipitated.

2a. PCR Analysis for Specific Regulatory Regions Containing Cis Sites or Transcribed Regions

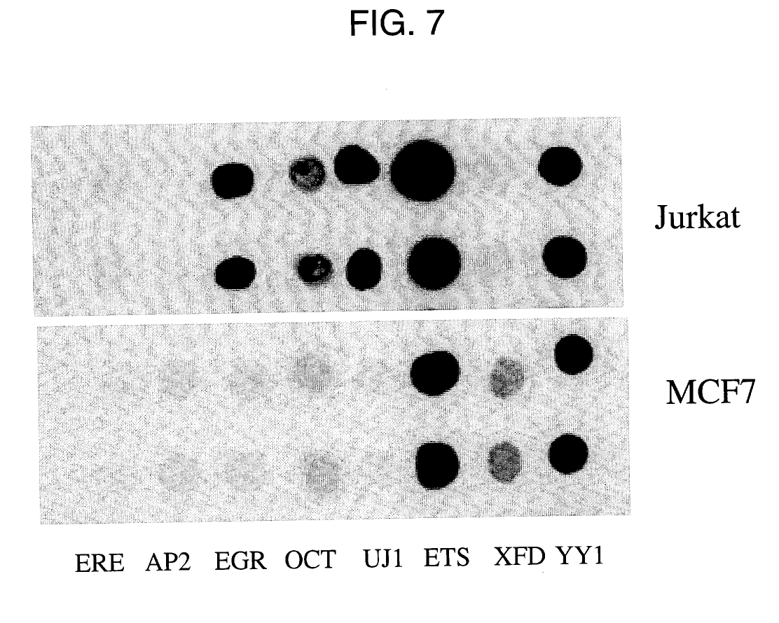

DNA primers specific for promoters, other regions upstream of known 5′ ends of genes, introns or exons were used in PCR amplification reactions for 20-25 cycles. Use of DNA primers specific for genes or genetic regions sufficiently far from their promoter regions were generally used to detect transcribed regions and ensure that signal detected was not due to polymerase sitting on the promoters without transcription. In some cases, PCR included ³²P-alpha-dNTP so that the amplified products could be detected by autoradiography of gels loaded with the reaction contents after incubation. In these experiments, it was important to determine that the “no antibody control” did not generate a PCR product. In some cases, a fraction of the non-precipitated cross-linked chromatin was processed in parallel as positive controls. Since signal from total chromatin should be equal between the two cell types or treatments being compared, any differences were also used to normalize the data. As seen in FIG. 7, bands corresponding to specific sequences containing the c-FOS (cellular gene corresponding to FBJ murine osteosarcoma virus oncogene), ER (estrogen receptor), c-ERBB2 (cellular gene corresponding to avian erythroblastosis virus oncogene v-erbB), histone H3 and LEF-1 (lymphoid enhancer factor 1) promoters were consistently observed after precipitation with antibodies directed against transcription-related proteins TFIIB (RNA polymerase II transcription factor B), TFIIEβ (RNA polymerase II transcription factor E, subunit β), AcH3 (acetylated histone H3), TBP (TATA-box binding protein) and CBP (CREB-binding protein). However, some of the band intensities were significantly greater in samples from one cell type or the other, indicating differential binding involving these promoters and proteins. For example, ER and c-ERB were bound (active) at higher levels in MCF7 cells, while the LEF-1 promoter was bound at much higher levels in Jurkat cells. These results were consistent with RNA expression studies involving the same genes and cell types.

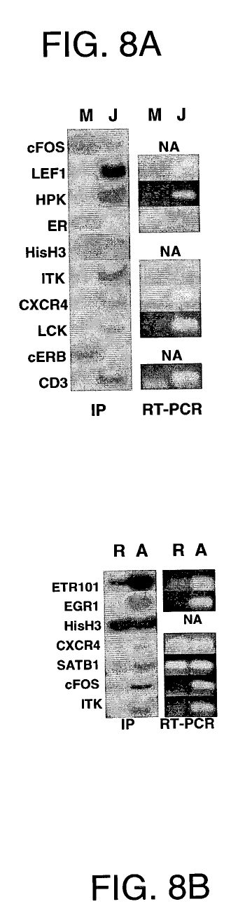

Immunoprecipitation of chromatin with antibodies against RNA polymerase II again showed that profiling of proteins associated with transcription could be carried out (see FIG. 8A). DNA sequences comprising c-FOS, ER and c-ERBB were found to be associated with polymerase at significantly higher levels in MCF7 vs. Jurkat cells, while DNA sequences comprising LEF1, HPK, ITK, CXCR4, LCK and CD3 were associated with polymerase at higher levels in the Jurkat cells. For 7 of the 10 genes that were also examined by RNA analysis using RT-PCR, the results with the two methods were in total agreement.

Profiling in the same manner by detection of DNA binding by RNA polymerase II in resting vs. activated Jurkat cells again showed that some genes were associated with polymerase at a higher frequency following cell activation (FIG. 8B). These included ETR101, EGR1, SATB1, cFOS and ITK, all of which agreed with concomitant RNA analysis except for SATB1. In the case of SATB1, it is possible that higher levels of transcription occurred in the activated state, but the RNA was turning over just as rapidly so that differential RNA expression was not detectable. In the case of regulatory element profiling, the differential gene expression detected was consistent with T cell activation.

2b. Quantitative PCR Analysis to Detect Transcribed Genes Using SYBR Green

DNA primers for gene-specific promoter regions, introns and exons were used to amplify immunoprecipitated DNA in a reaction containing Brilliant SYBR green Q-PCR master mix (Stratagene), 200 nM primers and immunoprecipitated chromatin template obtained from various cell populations. PCR reactions were performed and fluorescence accumulation was tracked using the ABI 7700 Sequence Detector and corresponding software. Cycling conditions were as follows: 95° C. for 10 minutes to activate the polymerase, and then 40 cycles consisting of 95° C. for 15 seconds, 60° C. for 15 seconds, and 72° C. for 30 seconds. Relative values, representative of the starting amount of immunoprecipitated DNA in each reaction, were assigned to each well using a standard curve and the ABI 7700 software. These values were normalized using the signals obtained from reactions with total chromatin corresponding to each preparation, to total DNA concentrations determined by use of Picogreen (Molecular Probes), or to values obtained with housekeeping genes (e.g., ubiquitin C, cyclophilin A, GAPDH, and HPRT). Values were also adjusted by subtracting out the signals generated with the “no antibody” controls.

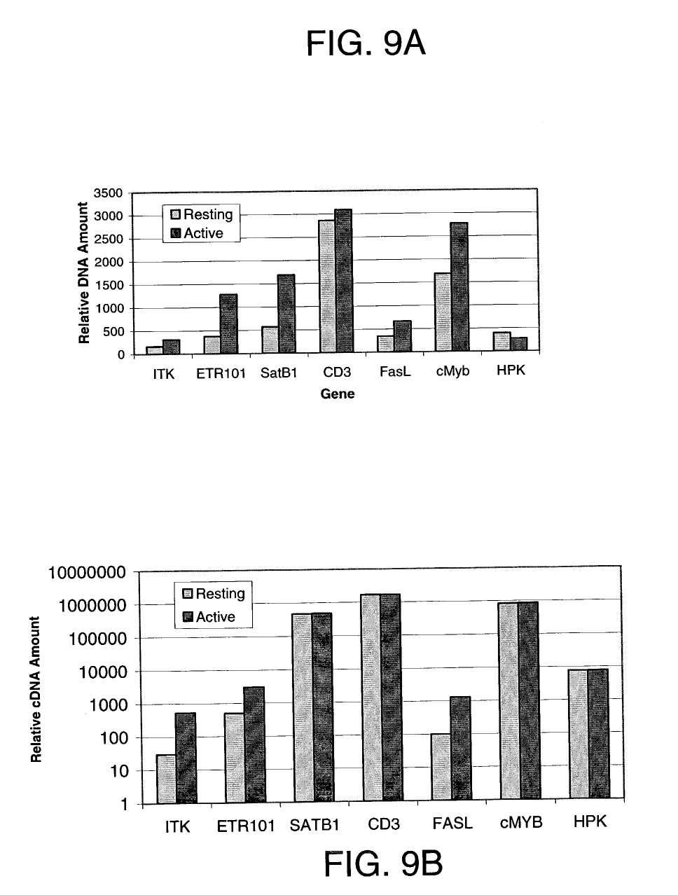

As shown in FIG. 9, Q-PCR analysis of chromatin immunoprecipitated with antibody against RNA polymerase II detected differential binding of this protein to DNA corresponding to several genes in resting vs. activated Jurkat cells. These genes included ITK, ETR101, SatB1, FasL and c-Myb, all genes likely associated with T cell activation. Comparison with RNA analysis by RT-PCR showed concordance between the two methods in the ITK, ETR101, and FasL genes, but SatB1 and c-Myb were not differential by RNA analysis. For ITK, ETR101 and SatB1, these results are in agreement with the results obtained by PCR and gel analysis shown in FIG. 8B. Again, since RNA analysis detects steady state levels of RNA, it is likely that the profiling method of the present invention can detect differential transcription not always detectable by RNA analysis.

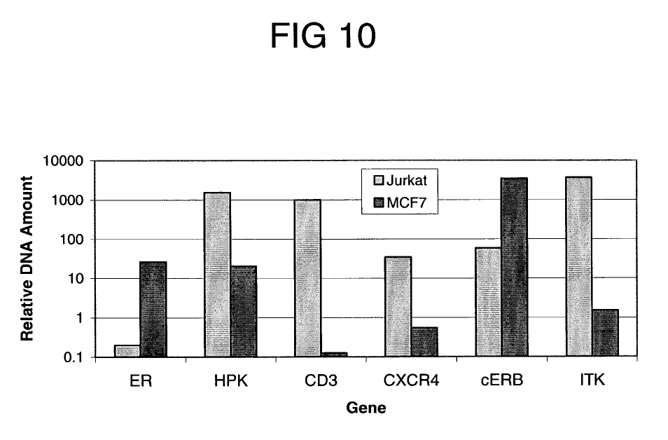

Similarly, element profiling by Q-PCR of pol II-precipitated DNA in Jurkat vs MCF-7 cells showed transcription at significantly higher levels in Jurkat cells for genes HPK, CD3, CSCR4 and ITK, while ER and c-ERB were transcribed at higher levels in MCF-7 (FIG. 10). These results agree completely with those shown in FIG. 8A from analysis using PCR and gel autoradiography.

3. Analysis of Precipitated DNA by Library Formation and Sequencing

A library of short (˜30-50 bp) fragments was generated from the immunoprecipitated DNA using the method developed by Singer et al., 1997 and described above (Example 2.A.2). DNA fragments were selected in the 80-90 bp range so that genomic DNA sequences in the center were about 40-50 bp. Inserts of the library were concatemerized by ligation in the presence of a double-stranded “adapter” DNA, resulting in concatamers of 15-20 fragments. Concatamers were amplified with primers corresponding to the adapter sequence and cloned into the CloneAmp vector (Invitrogen). Inserts of transformed bacteria were directly amplified by PCR and sequenced using standard sequencing methods known to those in the art. Sequenced DNA fragments were mapped on the human genome using the Human Genome Browser (University of California, Santa Cruz).

Table 2 shows examples of genes found to be associated with the transcription-related enzyme, RNA polymerase II, in MCF7 cells that were either treated with estradiol or mock-treated. In this analysis, of the 427 sequences that could be mapped in the human genome, 411 (96%) mapped to a known gene, EST (expressed sequence tag), mRNA, or predicted gene. In addition, a number of these genes, e.g., GREB 1, were detected multiple times, confirming their association with the transcription process. In the case of GREB 1, this gene was previously reported to be expressed in human breast cancer (Ghosh et al., 2000, Cancer Res., 60:6367).

TABLE 2

| MCF7 cells | MCF7 cells treated with Estradiol |
|---|---|
| GREB1, GREB1 protein | GATA3, GATA binding protein 3 |
| isoform a | |
| CELSR1, cadherin EGF LAG | IGSF3, immunoglobulin superfamily, |
| seven pass G-type receptor 1 | member 3 |
| HMG20A, high-mobility group | HDAC1, histone deacetylase 1 |
| 20A | |
| KCNG2, potassium voltage- | IDH1, isocitrate dehydrogenase 1 |
| gated channel subfamily G | (NADP+), soluble |
| MAP3K4, MAP/ERK kinase | KIP3, DNA-dependent protein kinase |
| kinase 4, isoform a | catalytic |
| PCTK1, PCTAIRE protein | MAPK311, mitogen-activated protein |
| kinase 1 isoform a | kinase kinase kinase |
| PIBF1, progesterone-induced | POLR2A, DNA directed RNA |
| blocking factor 1 | polymerase II polypeptide A |
| RGS3, regulator of G-protein | MTIF3, mitochondrial translational |
| signaling 3 isoform 3 | initiation factor 3 |
| SH3BP4, SH3-domain binding | PISD, phosphatidylserine decarboxylase |
| protein 4 | |

(Note that for each gene, first the gene symbol is given, followed by the descriptive name)

4. Comparative Analysis of Immunoprecipitated DNA by Subtractive Hybridization, Cloning and Sequencing

DNA was isolated from immunoprecipitated regulatory complexes originating from both resting Jurkat cells and from PMA/ionomycin-activated Jurkat cells (as described in Example 1 above). With both cell types, immunoprecipitation was carried out using a polyclonal antibody against RNA polymerase II (Santa Cruz Biotechnology). Each population of DNA molecules was then tagged by ligating to their ends double-stranded oligonucleotides (“adapters”), where the adapters were different between the two cell populations. For example, one of the adapters had the sequence: 5′-gcggtgacccgggagatctgaattc-3′ (SEQ ID NO:1) annealed to an a sequence 5′-gaattcagatc-3′ (SEQ ID NO:2). Another adapter had the sequence: 5′-cttcccagttccaggatccaattac-3′ (SEQ ID NO:3) annealed to a sequence: 5′-gtaattggatc-3′ (SEQ ID NO:4). The adapters for the DNA molecules isolated from one cell population were also labeled with biotin. The two DNA samples were then mixed together at a ratio such that the biotinylated fragments (called driver) were present in a 5-10-fold excess over the unbiotinylated fragments (called tester). Samples were incubated at 50° C. for 48-72 hours to allow annealing of complementary sequences. Hybrids containing at least one biotinylated strand were removed by use of streptavidin-coated magnetic beads (Roche Molecular Biochemicals). Hybrid isolation was carried out a second time to ensure complete capture of duplexes. The resulting supernatant after the magnetic separation was subjected to a second subtraction using a fresh addition of driver sequences. The final doubly-subtracted sample was then PCR-amplified using primer sequences specific for the tester adapter, and used in library construction, cloning and sequencing as described above for non-subtracted sequences (Example 3.A.3). Sequences were mapped to the human genome using the Human Genome Browser (University of California, Santa Cruz) and the Ensembl Browser (Sanger Institute).

Table 3 presents the results of the experiments as described in this example. Table 3, Left, shows genes identified in DNA fragments from resting Jurkat cells that had been subtracted using DNA fragments from activated Jurkat cells, where the DNA fragments were originally found to be associated with RNA polymerase II as a result of immunoprecipitation. Similarly, Table 3, Right, shows genes identified in DNA fragments from activated Jurkat cells that had been subtracted using DNA fragments from resting Jurkat cells. These results demonstrate that specific genes can be identified in DNA populations isolated as a result of association with transcription-related proteins such as RNA polymerase, and that these genes remain after subtraction with DNA molecules obtained from a related type of cell.

TABLE 3

| Resting minus | Activated minus |
|---|---|
| Activated Jurkat Cells | Resting Jurkat Cells |
| MLLT7, myeloid/lymphoid or | NAB2, NGF1-A binding protein 2 |
| mixed-lineage leukemia | |
| Eu-HMTasel, euchromatic | PML, promyelocytic leukemia protein, |
| histone methyltransferase 1 | isoform 1 |
| DNMT2, DNA (cytosine-5-)- | RREB 1, ras responsive element |
| methyltransferase 2 | binding protein 1 |
| ATF5, activating transcription | ASK, activator of S phase kinase |
| factor 5 | |
| TRAF2, TNF receptor- | IDH2, isocitrate dehydrogenase 2 |
| associated factor 2 isoform 2 | (NADP+) |
| IL22R, interleukin 22 receptor | ECE1, endothelin converting enzyme 1 |
| SMARCA4, SWI/SNF-related | ZNF145, zinc finger protein 145 |
| matrix-associated/LTR | (Kruppel-like, expressed) |
| POLR2J2, DNA directed RNA | SRRM2, splicing coactivator subunit |
| polymerase II polypeptide | SRm300 |

(Note that for each gene, first the gene symbol is given, followed by the descriptive name)

### Example 4

This example describes methods of the invention as applied to the global profiling of gene expression using pheochromocytoma 12 (PC12) cells.

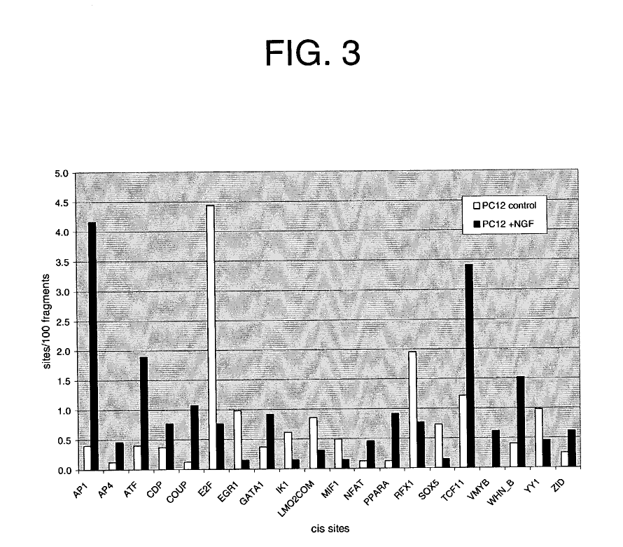

Global regulatory element activity profiling was carried out using nuclear extracts from cells that were either untreated or treated with NGF-beta. Known transcription factor binding sites present in the DNA sequences were counted as described for Jurkat cells. The data generated corresponding to a partial global profile are presented in FIG. 3. Bars indicate the percentage of DNA fragments containing selected cis sites that were isolated in binding reactions containing nuclear extracts from either untreated (white bars) or NGFbeta-treated (black bars) PC12 cells. It can be seen that NGF treatment led to an increase in binding activity for AP 1, ATF and TCF11 (among others), while other activities, for example, E2F and RFX1, were reduced after NGF treatment. This analysis suggests that genes regulated by AP1, ATF or TCF11 are activated upon NGF treatment, while genes regulated by E2F and RFX1 are repressed upon NGF treatment.

The results in PC12 cells involving the activity of specific cis site-transcription factor complexes and their ability to regulate gene expression are related to diseases involving neuronal cell death and regeneration. For example, in the PC 12 model, AP-1 expression is associated with neurite outgrowth and protection from apoptosis (Dragunow et al, 2000, Brain Res. Mol Brain Res., 83:20-33). Certain human neurodegenerative diseases can also involve either acute injury or chronic neuronal changes. Thus, the profiling of the present invention provides a real world application for identifying the regulatory effects of disease-related molecules.

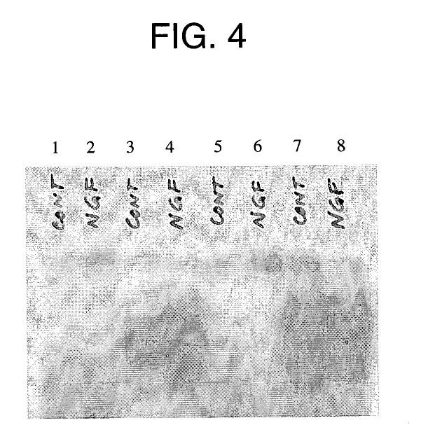

Confirmation of the increase in the transcription factor AP-1 binding activity was performed by electrophoretic mobility shift assay (EMSA). Nuclear extracts obtained from PC cells either treated with NGF-beta or untreated were combined in separate binding reactions. Each reaction also contained a ³²P-labeled oligonucleotide comprising a binding site for a specific transcription factor. As shown in FIG. 4 (lanes 3 and 4), a significant increase in the gel-shifted material (DNA-protein complexes) was observed in the NGF-treated cells when the oligonucleotide was specific for the AP-1 binding site. In contrast, when the oligonucleotide was specific for the OCT1 binding site (lanes 7 and 8), no increase in gel-shifted material was observed. These results further demonstrate that AP-1 is increased in binding activity to its cis site sequence in PC12 cells treated with NGF-beta. The data also demonstrate that OCT1 cis site-transcription factor complexes are present in both cell populations, but are not differential between the NGF-treated and untreated cells.

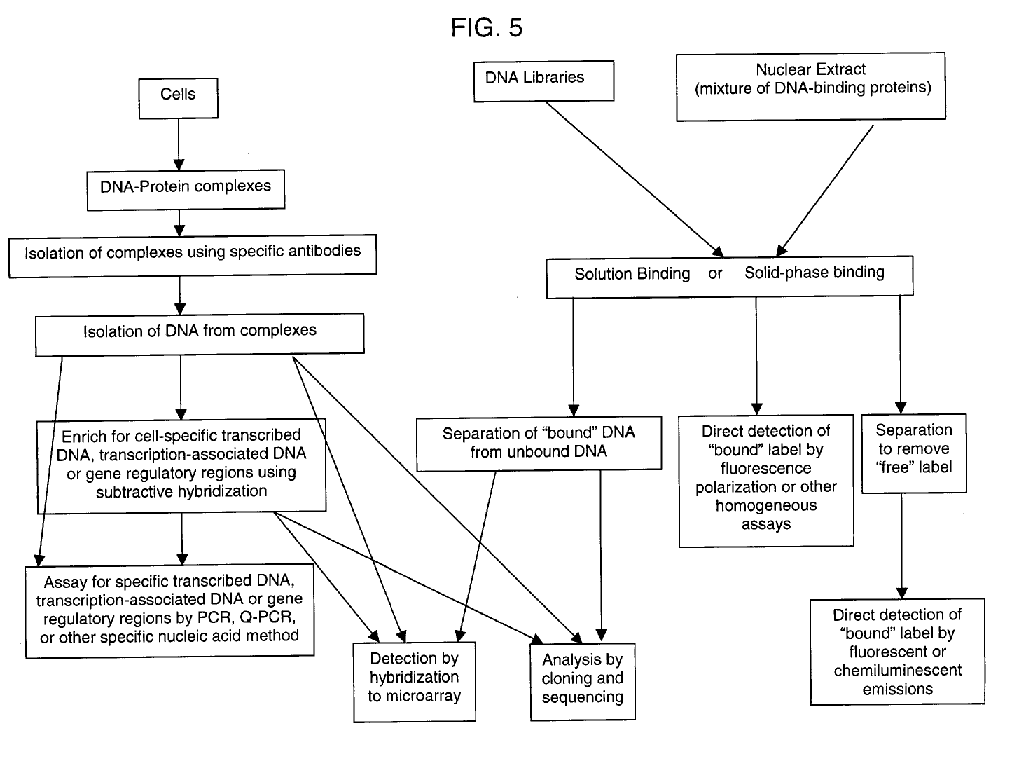

These results also illustrate the feasibility as well as the usefulness of determining global gene regulatory element activity profiles involving quantitative levels of cis site-nucleic acid binding factor activities within cell populations. These profiles can then be compared between different cell populations to discern differences in gene expression important in overall genetic and phenotypic changes. As is clear to those skilled in the art, identification of such changes, as determined by the global profiling of the present invention, is useful in many applications in medicine, such as in determining the effects of compounds on gene regulation, and recognition of disease states in cells. FIG. 5 provides a schematic of the profile determining process of the present invention.

The contents of all patents, patent applications, published PCT applications and articles, books, references, reference manuals, abstracts, and internet websites cited herein are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

What is claimed is:

1. A method of determining a global gene regulatory element profile of cells, comprising:
(a) obtaining from at least one cell, or from cellular contents obtained from at least one cell, a plurality of one or more types of gene regulatory element complexes formed between cellular nucleic acid and associated protein components, said complexes comprising: (i) nucleic acid molecules and nucleic acid binding proteins; (ii) nucleic acid binding proteins and regulatory proteins; (iii) nucleic acid molecules, nucleic acid binding proteins and regulatory proteins; (iv) nucleic acid molecules, nucleic acid binding proteins, regulatory proteins and co-regulatory proteins; or (v) combinations thereof, under conditions conducive to the formation of said complexes;
(b) detecting the components of the complexes that are formed; and
(c) identifying one or more of the nucleic acid molecule, nucleic acid binding protein, regulatory protein, or co-regulatory protein components comprising the complexes so as to determine (i) a global gene regulatory element profile of the cells or (ii) a global analysis of transcription events occurring in the cells.

2. The method according to claim 1, wherein the gene regulatory element complexes are produced within the at least one cell and isolated therefrom.

3. The method according to claim 1, wherein the gene regulatory element complexes are produced outside of a cell by contacting a source of cellular nucleic acid sequences with a source of cellular proteins under conditions allowing for generation of the complexes.

4. The method according to claim 1, further comprising identifying transcribed regions regulated by the gene regulatory element complexes.

5. The method according to claim 1, wherein the complexes are formed inside living cells prior to isolation.

6. The method according to claim 1, wherein the complexes are formed under cell-free binding conditions prior to isolation.

7. The method according to claim 1, wherein the complexes are formed in solution.

8. The method according to claim 1, wherein the complexes are immobilized on localizing surfaces.

9. The method according to claim 1, wherein the cells are prokaryotic cells or eukaryotic cells.

10. The method according to claim 9, wherein the cells are selected from mammalian cells, vertebrate cells, invertebrate cells, plant cells, fungal cells, insect cells, protozoan cells, algal cells, yeast cells, Archaebacterial cells, and bacterial cells.

11. The method according to claim 9, wherein the cells are selected from single cells, cloned cells, homogeneous populations of cells, semi-purified cells, fully-purified cells, cells from tissues or portions thereof, cells from organs or portions thereof, or cells from whole organisms or portions thereof.

12. The method according to claim 11, wherein the cells comprise mixtures of different cell populations.

13. The method according to claim 1, wherein the nucleic acid molecules comprise one or more cis sites.

14. The method according to claim 13, wherein the one or more cis sites comprise associated transcribed regions.

15. The method according to claim 1, wherein the nucleic acid molecules comprise one or more gene regulatory sequences.

16. The method according to claim 13, wherein the nucleic acid molecules comprising one or more cis sites are obtained from cells, a preparation of genomic nucleic acid molecules, cloned nucleic acid sequences, or a library of synthetically prepared nucleic acid molecules.

17. The method according to claim 13, wherein regulatory or co-regulatory proteins are stably associated with the one or more cis sites comprising the nucleic acid molecules of the complexes.

18. The method according to claim 17, wherein the stable association of the regulatory or co-regulatory proteins and the cis sites comprising the nucleic acid molecules of the complexes results from one or more of chemical cross-linking, biological cross-linking, ultraviolet light cross-linking, or cleavable linker interactions.

19. The method according to claim 18, wherein the cross-linking is reversible.

20. The method according to claim 1, wherein the nucleic acid molecule and protein components comprising the complexes are obtained from a total cell extract, a nuclear extract, a cytoplasmic extract, a mitochondrial extract, a choloroplast extract, or a subcellular extract of the cells.

21. The method according to claim 1, further comprising performing steps (a)-(c) to determine a global gene regulatory element profile for (i) different cells, or (ii) two or more populations of cells, and comparing the profiles.

22. The method according to claim 1, further comprising selecting regulatory protein or nucleic acid molecule components of the complexes that bind to molecules involved in gene expression or transcription.

23. The method according to claim 22, wherein the molecules involved in gene expression or transcription comprise transcription factors.

24. The method according to claim 22, wherein the molecules involved in gene expression or transcription comprise promoter-associated factors.

25. The method according to claim 22, wherein the molecules involved in gene expression or transcription comprise enhancer-associated factors.

26. The method according to claim 1, wherein the regulatory proteins detected and identified from the complexes include general transcription factors, specific transcription factors that regulate subsets of genes, transcription-associated proteins, or co-regulatory proteins.

27. The method according to claim 26, wherein the transcription associated protein is polymerase.

28. The method according to claim 1, wherein the gene regulatory element profile provides the identification of a difference between gene expression or regulation of cells in one cellular metabolic state and gene expression or regulation of cells in a second cellular metabolic state.

29. The method according to claim 1, wherein the gene regulatory element profile provides the identification of a difference between gene expression or regulation of diseased cells and gene expression or regulation of non-diseased cells.

30. The method according to claim 1, wherein the gene regulatory element profile provides the identification of a difference between gene expression or regulation of normal cells and gene expression or regulation of abnormal cells.

31. The method according to claim 1, wherein the gene regulatory element profile provides the identification of a difference between gene expression or regulation of cells in one cellular physiologic state and gene expression or regulation of cells in a second cellular physiologic state.

32. The method according to claim 1, wherein the gene regulatory element profile provides the identification of a difference between gene expression or regulation of cells treated with an exogenous substance or agent and gene expression or regulation of untreated cells.

33. The method according to claim 32, wherein the exogenous substance or agent comprises a drug or chemical.

34. The method according to claim 13, further comprising identifying nucleic acid regulatory regions in cis site-containing nucleic acid molecules comprising the complexes by a method selected from nucleic acid amplification, nucleic acid sequencing, nucleic acid hybridization, or a combination thereof.

35. The method according to claim 1, further comprising identifying the nucleic acid sequences of the nucleic acid molecules bound to protein in the complexes by a method comprising the steps of:
a) denaturing the nucleic acid sequences comprising the complexes;
b) binding the denatured nucleic acid sequences to detectably labeled nucleic acid molecules of known sequence; and
c) identifying the nucleic acid molecules from the complex that were previously bound to protein by their binding to the detectably labeled nucleic acid molecules of known sequence.

36. The method according to claim 35, further comprising the step of d) sequencing the identified nucleic acid molecules.

37. The method according to claim 35, further comprising determining cis site motifs in the identified nucleic acid molecules associated with protein in the complexes.

38. The method according to claim 35, further comprising quantifying the nucleic acid molecules bound to the detectably labeled nucleic acid molecules of known sequence by determining intensity of the detectable label.

39. The method according to claim 38, wherein the detectable label comprises a fluorescent label, a radioactive label, an enzymatic label, or a chemiluminescent label.

40. The method according to claim 1, further comprising identifying the proteins comprising the complexes by a method selected from immunodetection, receptor-ligand binding, chemical methods, peptide sequencing, or array binding.

41. The method according to claim 40, wherein immunodetection is performed using antibodies directed toward one or more proteins of the complexes.

42. The method according to claim 40, wherein array binding is performed by (i) binding the proteins from the complexes onto an array comprising antibodies directed toward the protein in the complex, or (ii) binding nucleic acid from the complexes onto an array comprising nucleic acid molecules of known sequence.

43. The method according to claim 1, wherein identifying the nucleic acid molecules in the isolated complexes optionally comprises cloning fragments of the nucleic acid molecules into vectors, and (i) hybridizing the cloned nucleic acid molecules to nucleic acid probes of known sequence, or (ii) sequencing the cloned nucleic acid molecules.

44. The method according to claim 1, wherein analysis of the nucleic acid molecules in the isolated complexes comprises amplifying the nucleic acid molecules, or fragments thereof, subjecting the amplified nucleic acid molecules, or fragments thereof, to gel electrophoresis and observing amplicons of the expected size or sequences of the expected type.

45. The method according to claim 1, wherein analysis of the nucleic acid molecules in the isolated complexes comprises hybridizing the amplified nucleic acid molecules, or fragments thereof, to macroarrays or microarrays containing thereon known nucleic acid sequences.

46. The method according to claim 44 or claim 45, wherein the known nucleic acid sequences comprise cis sites, transcription regulatory regions, known transcribed regions, or predicted transcribed regions.

47. The method according to claim 44, wherein amplifying the nucleic acid molecules comprises a method selected from polymerase chain reaction (PCR), quantitative PCR (Q-PCR), ligation-mediated PCR (LM-PCR), transcription-mediated amplification, rolling circle amplification, or ligase chain reaction.

48. The method according to claim 1, further comprising directly sequencing the nucleic acid molecules, or fragments thereof, comprising the complexes, and evaluating the sequences obtained.

49. The method according to claim 1, wherein the nucleic acid molecules, or fragments thereof, comprising the complexes are isolated by binding to nucleic acid probes of known sequence or to arrays having bound thereto nucleic acid probes of known sequence.

50. The method according to claim 49, wherein the isolated nucleic acid molecules, or fragments thereof, are used as templates to synthesize a library of nucleic acid fragments comprising a selected population of nucleic acid sequences bound to protein in the complexes.

51. The method according to claim 50, wherein the library represents a portion of the bound sequences or sequences that are contiguous to the bound sequences.

52. The method according to claim 1 or claim 50, wherein the nucleic acid molecules identified or isolated from the complexes are subjected to subtractive hybridization.

53. The method according to claim 52, wherein the subtractive hybridization results in one or more of (i) enriching for nucleic acid sequences that are bound by a specific nucleic acid binding protein, regulatory protein, or co-regulatory protein in the complex; (ii) removing sequences common to regulatory element complexes from two or more different cells or populations of cells; (iii) enriching for sequences differentially present in regulatory element complexes from one cell or population of cells versus another cell or population of cells; (iv) enriching for sequences common to regulatory element complexes from two or more types of cells or cell populations; or (v) removing sequences present in regulatory element complexes from one cell or cell population versus another cell or cell population.

54. The method according to claim 1, further comprising comparing the global gene regulatory element profiles from cells comprising two or more different cell populations.

55. The method according to claim 54, wherein the two or more different cell populations being compared comprise different cell types within the same organism, the same cell type between different organisms, normal and diseased cells of the same type, normal and transformed cells of the same type, cells at different stages of differentiation, cells at different stages of development, treated cells and untreated cells, cells exposed to one compound and cells exposed to a second compound, cells exposed to an external condition and unexposed cells, or cells exposed to an internal condition and unexposed cells.

56. The method according to claim 1, wherein the complexed nucleic acid molecules comprise DNA, RNA, single-stranded DNA, single-stranded RNA, double-stranded DNA, double-stranded RNA, genomic DNA, complementary DNA, DNA complementary to RNA, modified DNA, or modified RNA.

57. A method of determining a global gene regulatory element profile of cells, comprising:
(a) isolating from two or more different cell populations, or from cellular contents obtained from the cell populations, a plurality of one or more types of gene regulatory element complexes, said complexes formed between cellular nucleic acid molecules and associated protein components;
(b) detecting the nucleic acid molecule or associated protein components of the complexes that are formed; and
(c) identifying one or more of the nucleic acid molecule or associated protein components comprising the complexes so as to determine a global gene regulatory element profile of the cells or a global analysis of transcription events occurring in the cells.

58. The method according to claim 57, wherein the gene regulatory element complexes are produced within the cells and isolated therefrom.

59. The method according to claim 57, wherein the gene regulatory element complexes are produced outside of cells by contacting a source of cellular nucleic acid sequences with a source of cellular proteins under conditions allowing for generation of the complexes.

60. The method according to claim 1 or claim 57, wherein the global gene regulatory element profile is further combined with a technique selected from RNA analysis, proteomics analysis, transcription factor characterization, or transcription factor assay to elucidate ongoing regulatory and transcription events involving gene expression in cells.

61. A method of globally profiling gene regulatory element activity of cells, comprising:
(a) obtaining from cells, or from cellular contents obtained from the cells, a plurality of one or more types of gene regulatory element complexes formed between cellular nucleic acid molecules and associated protein components, said components comprising: (i) nucleic acid molecules and nucleic acid binding protein complexes; (ii) nucleic acid binding protein and regulatory protein complexes; (iii) nucleic acid molecules, nucleic acid binding protein and regulatory protein complexes; (iv) nucleic acid molecules, nucleic acid binding protein, regulatory protein and co-regulatory protein complexes; or (v) combinations thereof, under conditions conducive to the formation of said complexes;
(b) isolating one or more of the protein components of the complexes using one or more affinity reagents that bind specifically to (i) the nucleic acid molecule component; (ii) the nucleic acid binding protein component; (iii) the regulatory protein component; (iv) the co-regulatory protein component; or (v) a combination thereof; and
(c) identifying one or more of the nucleic acid molecule components, nucleic acid binding protein components, regulatory protein components, or co-regulatory protein components comprising the complexes so as to determine a global gene regulatory element profile of the cells.

62. The method according to claim 61, wherein the gene regulatory element complexes are produced within the cells and isolated therefrom.

63. The method according to claim 61, wherein the gene regulatory element complexes are produced outside of cells by contacting a source of cellular nucleic acid sequences with a source of cellular proteins under conditions allowing for generation of the complexes.

64. The method according to claim 61, wherein the one or more affinity reagents is selected from polyclonal antibodies, or binding fragments thereof, monoclonal antibodies or binding fragments thereof, intrabodies, single chain antibodies, or ligand-binding receptor proteins.

65. The method according to claim 61, wherein the one or more affinity reagents binds to a general transcription factor.

66. The method according to claim 61, wherein the one or more affinity reagents binds to a specific transcription factor.

67. The method according to claim 61, wherein the one or more affinity reagents binds to proteins involved in active transcription

68. The method according to claim 61, wherein the one or more affinity reagents binds to one or more nucleic acid molecules of the complexes.

69. The method according to claim 68, wherein the one or more affinity reagents is selected from nucleic acid aptamers or nucleic acid probes.

70. The method according to claim 61, wherein (i) the isolated nucleic acid molecule components of the complexes are identified by determining their nucleic acid sequences; (ii) the isolated protein components of the complexes are identified by determining their amino acid sequences; or (iii) a combination of (i) and (ii).

71. The method according to claim 70, wherein the identified nucleic acid sequences are further mapped on the appropriate genome using nucleic acid sequence databases.

72. The method according to claim 61, wherein the complexes of step (a) are formed in solution, on a solid support, in semi-solid medium, in gels, in column matrices, or in polymer formulations.

73. The method according to claim 72, wherein the solution is an aqueous solution, an organic solution, or an inorganic solution.

74. The method according to claim 61, wherein, following step (a), the nucleic acid molecule and associated protein complexes are separated from unbound cellular material.

75. A method of globally profiling gene regulatory activity of cells, comprising:
(a) obtaining nucleic acid molecule and protein complexes formed (i) within cells under conditions conducive to the formation of the complexes, or (ii) extracellularly from cellular nucleic acids and cellular proteins contacted under conditions allowing for production of the complexes;
(b) isolating nucleic acid molecules from the complexes;
(c) enriching the nucleic acid molecules for cell-specific transcribed nucleic acid molecules; and
(d) determining the nucleic acid molecules that are specifically transcribed.

76. The method according to claim 75, further comprising the step of (e): identifying one or more of the proteins that comprise the complexes.

77. The method according to claim 75, wherein the nucleic acid comprising the complexes is DNA or RNA.

78. The method according to claim 75, wherein the complexes are obtained using antibodies directed against a protein comprising the complex.

79. The method according to claim 75, wherein the nucleic acid is identified by hybridization to nucleic acid probes, by binding to specific cis site-containing or regulatory-sequence-containing nucleic acid sequences, or by binding to nucleic acid molecules of known sequence or to immunoreactive agents arranged in an array.

80. The method according to claim 75, wherein the nucleic acid is isolated from the complexes using one or more of protease-digestion, phenol extraction and ethanol precipitation.

81. The method according to claim 75, wherein, in step (c), the cell-specific transcribed nucleic acid is enriched by subtractive hybridization to result in one or more of (i) enriching for nucleic acid sequences that are bound by a specific nucleic acid binding protein, regulatory protein, or co-regulatory protein in the complex; (ii) removing sequences common to regulatory element complexes from two or more different cells or populations of cells; (iii) enriching for sequences differentially present in regulatory element complexes from one cell or population of cells versus another cell or population of cells; (iv) enriching for sequences common to regulatory element complexes from two or more types of cells or cell populations; or (v) removing sequences present in regulatory element complexes from one cell or cell population versus another cell or cell population.

82. The method according to claim 75, wherein the specifically transcribed nucleic acid is determined by an amplification method selected from polymerase chain reaction (PCR), quantitative PCR (Q-PCR), ligation-mediated PCR, rolling circle amplification, transcription-mediated amplification and ligase chain reaction.

83. A method of globally profiling gene regulatory activity of cells, comprising:
(a) immunoprecipitating a plurality of regulatory element complexes comprising nucleic acid molecules and bound proteins from one or more cells or populations of cells;
(b) analyzing the immunoprecipitated nucleic acid molecules for the presence of regulatory regions comprising cis sites or transcribed regions to obtain a global profile of gene regulatory activity; and
(c) comparing the global profile of gene regulatory activity obtained in step (b) with global profiles of gene regulatory activity from different cells or cell populations to determine differences in gene expression or regulation in the different cell populations.

84. The method according to claim 83, wherein the regulatory element complexes are produced within the cells and isolated therefrom.

85. The method according to claim 83, wherein the regulatory element complexes are produced outside of the cells by contacting a source of cellular nucleic acid sequences with a source of cellular proteins under conditions allowing for generation of the complexes.

86. The method according to claim 83, wherein the cells are subjected to fixative prior to step (a).

87. The method according to claim 83, wherein cells are subjected to a cross-linking agent prior to step (a).

88. The method according to claim 83, wherein immunoprecipitation is performed using antibodies directed against transcription related proteins.

89. The method according to claim 88, wherein transcription related proteins are selected from RNA polymerase II, RNA polymerase II transcription factor B (TFIIB), RNA polymerase II transcription factor E, subunit β (TFIIEβ), acetylated histone H3 (AcH3), TATA-box binding protein (TBP) and CREB-binding protein (CBP).

90. The method according to claim 83, wherein the analyzing step (b) is performed using polymerase chain reaction (PCR).

91. The method according to claim 90, wherein the primers in the polymerase chain reaction (PCR) are specific for promoter sequences, intronic sequences, exonic sequences, enhancer sequences, sequences 5′ to promoter sequences, sequences 5′ or 3′ to genes or a combination thereof.

92. The method according to claim 83, further wherein the analyzing step (c) comprises using quantitative PCR (Q-PCR) to detect transcribed genes.

93. The method according to claim 83, further comprising identifying the protein components that are complexed with the nucleic acids.

94. A method for globally determining differences in gene regulatory element activity between cells, comprising:
(a) isolating from a first population of cells a plurality of gene regulatory complexes comprising nucleic acid molecule components and associated protein components;
(b) analyzing (i) the nucleic acid molecule components of the complexes of step (a) to determine the presence of cis sites or regulatory regions; (ii) the protein components of the complexes of step (a) to identify the proteins as nucleic acid binding proteins, regulatory proteins, or co-regulatory proteins; or a combination of (i) and (ii);
(c) isolating from a second population of cells a plurality of gene regulatory complexes comprising nucleic acid molecule components and associated protein components;
(d) analyzing (i) the nucleic acid molecule components of the complexes of step (c) to determine the presence of cis sites or regulatory regions; (ii) the protein components of the complexes of step (c) to identify the proteins as nucleic acid binding proteins, regulatory proteins, or co-regulatory proteins; or a combination of (i) and (ii); and
(e) comparing the components of the complexes isolated from the first and second populations of cells to determine differences in gene regulatory element activity between the cell populations.

95. The method according to claim 94, wherein the gene regulatory element complexes are produced within the first and second populations of cells and isolated therefrom.

96. The method according to claim 94, wherein the gene regulatory element complexes are produced outside of the first and second populations of cells, by contacting a source of cellular nucleic acid sequences with a source of cellular proteins under conditions allowing for generation of the complexes for each population.

97. The method according to claim 94, wherein the first population of cells comprises cells selected from a first cell type, physiologic state, metabolic state, disease state, or drug-treated state, and the second population of cells comprises cells selected from a second cell type, physiologic state, metabolic state, disease state, or drug-treated state.

98. The method according to claim 94, wherein the first and second populations of cells comprise different cell types within the same organism, the same cell type between different organisms, normal cells and diseased cells of the same types, normal and transformed cells of the same types, cells at different stages of differentiation or development, cells treated with an exogenous material and untreated cells, cells exposed to two different compounds or molecules, cells exposed to a different external or internal condition and unexposed cells, cells exposed to two different external or internal conditions, or infected cells and uninfected cells.

99. A method of determining a global gene regulatory element profile in cells, comprising:
(a) obtaining from two or more different cell populations a plurality of one or more types of gene regulatory element complexes formed between cis site-containing or regulatory sequence-containing nucleic acid molecules and associated protein components; and
(b) detecting and analyzing one or more of the nucleic acid molecule or associated protein components comprising the complexes of the cell populations; and
(c) comparing the nucleic acid molecule or protein components from the cell populations so as to determine global gene regulatory element activity in the two or more cell populations, or a global analysis of transcription events occurring in the two or more cell populations.

100. The method according to claim 99, wherein the gene regulatory element complexes are produced within the cells and isolated therefrom.

101. The method according to claim 99, wherein the gene regulatory element complexes are produced outside of cells by contacting a source of cellular nucleic acid sequences with a source of cellular proteins under conditions allowing for generation of the complexes.

102. The method according to claim 99, wherein cis sites contained in the nucleic acid molecules of the complexes of the cell populations are identified by isolating the nucleic acid molecules, or fragments thereof, and determining cis site-containing nucleic acid sequences.

103. The method according to claim 99, wherein cis sites contained in the nucleic acid molecules of the complexes of the cell populations are identified by amplifying fragments obtained from the nucleic acid molecules of the complexes and obtaining overlapping or nonoverlapping fragments, wherein the obtained fragments are further size-selected and concatamerized for cloning and sequencing.

104. The method according to claim 103, wherein the fragments are about 50-100 base pairs in length.

105. The method according to claim 99, wherein the nucleic acid molecules, or fragments thereof, of the complexes of the cell populations are hybridized to probes having known nucleic acid sequences under conditions suitable for hybrid formation, wherein the sequence of a nucleic acid molecule, or fragment thereof, is determined following the formation of hybrids.

106. The method according to claim 105, wherein prior to hybridization, the nucleic acid molecules, or fragments thereof, are amplified.

107. The method according to claim 105, further comprising a detectable label to allow detection of hybridization complexes.

108. The method according to claim 107, wherein the detectable label comprises a radioactive label, an enzymatic label, a fluorescent label, or a chemiluminescent label.

109. The method according to claim 105, wherein hybridization is performed in solution, on macroarrays, or on microarrays.

110. The method according to claim 105, wherein hybrid complexes are detected by autoradiography, fluorimetry, luminometry, or phosphoimage analysis.

111. A method for globally profiling regulatory element activity of cells, comprising:
(a) obtaining from the cells, or from cellular contents obtained from the cells, a plurality of gene regulatory element complexes comprising cis site-containing nucleic acid molecules and associated protein components selected from (i) nucleic acid molecules and nucleic acid binding proteins; (ii) nucleic acid binding proteins and regulatory proteins; (iii) nucleic acid molecules, nucleic acid binding proteins and regulatory proteins; (iv) nucleic acid molecules, nucleic acid binding proteins, regulatory proteins and co-regulatory proteins; or (v) combinations thereof, under conditions conducive to the formation of said complexes;
(b) detecting the complexes;
(c) identifying (i) a nucleic acid sequence of one or more cis site-containing nucleic acid molecules comprising the complexes, (ii) an amino acid sequence of one or more nucleic acid binding proteins, regulatory proteins, or co-regulatory proteins comprising the complexes, or a combination of (i) and (ii); wherein identification of the nucleic acid components of the separated complexes comprises one or more of (1) sequencing the nucleic acid molecules or a portion thereof; (2) hybridizing the nucleic acid molecules to other known nucleic acid molecules; (3) preparing a recombinant library from the isolated nucleic acid molecules or portions thereof; (4) sequencing the library or a portion thereof; or (5) amplifying the nucleic acid sequences to determine if specific nucleic acid sequences are present in the isolated nucleic acid molecules so as to globally profile gene regulatory element activity in the cells.

112. The method according to claim 111, wherein the gene regulatory element complexes are produced within the cells and isolated therefrom.

113. The method according to claim 111, wherein the gene regulatory element complexes are produced outside of cells by contacting a source of cellular nucleic acid sequences with a source of cellular proteins under conditions allowing for generation of the complexes.

114. The method according to claim 111, wherein the complexes of step (b) and the sequences of step (c) are compared between a first cell type, cell population, cell state, or cell treatment and at least a second cell type, cell population, cell state, or cell treatment to globally profile gene regulatory element activity in the compared cells, or to compare global gene regulatory element activity profiles between the cells or cell populations.

115. The method according to claim 111, wherein the detecting step (b) comprises fluorescent polarization.

116. The method according to claim 111, wherein the detecting step (b) comprises direct detection comprising a fluorescent label or a chemiluminescent label.

117. The method according to claim 111, wherein the detecting step (b) comprises separating the complexes from exogenous material.

118. The method according to claim 1, claim 57, claim 99, or claim 111, wherein the complexes are separated from nucleic acid molecules and proteins not comprising the complexes before the detecting step (b).

119. The method according to claim 118, wherein the separation is performed by one or more of the methods selected from electrophoretic mobility shift assay (EMSA), capillary electrophoresis (CE), filtration, size-exclusion filtration, affinity purification, enzyme digestion and centrifugation.

120. The method according to claim 111, wherein the cis site-containing nucleic acid molecules are contacted with a surface comprising a macroarray or a microarray.

121. The method according to claim 111, wherein the cis site-containing nucleic acid molecules are obtained from cells, genomic nucleic acid, or a library of synthetically prepared nucleic acid molecules.

122. The method according to claim 111, wherein the cells are selected from the group consisting of mammalian cells, vertebrate cells, invertebrate cells, plant cells, fungal cells, insect cells, protozoan cells, algal cells, yeast cells, Archaebacterial cells, and bacterial cells.

123. The method according to claim 111, wherein the cells are selected from single cells, cloned cells, homogeneous populations of cells, semi-purified cells, fully-purified cells, cells from tissues or portions thereof, cells from organs or portions thereof, or cells from whole organisms or portions thereof.

124. A method of determining a global gene regulatory element activity profile of cells, comprising:
(a) isolating from at least one cell, or from cellular contents obtained from at least one cell, a plurality of one or more types of gene regulatory element complexes formed between cellular nucleic acid and associated protein components, said complexes comprising: (i) nucleic acid molecules and nucleic acid binding proteins; (ii) nucleic acid binding proteins and regulatory proteins; (iii) nucleic acid molecules, nucleic acid binding proteins and regulatory proteins; (iv) nucleic acid molecules, nucleic acid binding proteins, regulatory proteins and co-regulatory proteins; or (v) combinations thereof, under conditions conducive to the formation of said complexes;
(b) separating the one or more types of complexes from other complexes and/or from unbound components;
(c) identifying (i) the nucleic acid components of the separated complexes, or (ii) the protein components of the separated complexes; and
(d) combining activity information of at least two of the complexes to generate a global gene regulatory element activity profile for the cells.

125. The method according to claim 124, wherein the gene regulatory element complexes are produced within the cells and isolated therefrom.

126. The method according to claim 124, wherein the gene regulatory element complexes are produced outside of cells by contacting a source of cellular nucleic acid sequences with a source of cellular proteins under conditions allowing for generation of the complexes.

127. The method according to claim 124, wherein the nucleic acid molecules of the complexes are fragmented before separating the complexes.

128. The method according to claim 127, wherein the nucleic acid molecules are fragmented using sonication, restriction enzyme digestion, nuclease digestion, pH or elevation of temperature.

129. The method according to claim 124, wherein the separating at least one type or class of complexes comprises use of affinity reagents.

130. The method according to claim 129, wherein the affinity reagents include antibodies that recognize transcription-associated proteins.

131. The method according to claim 129, wherein the affinity reagents include nucleic acid probes that recognize transcription-associated nucleic acids.

132. The method according to claim 130, wherein the transcription-associated proteins are selected from general transcription factors, specific transcription factors that regulate subsets of genes, transcription-associated proteins, or co-regulatory proteins.

133. The method according to claim 132, wherein the transcription associated proteins are polymerases.

134. The method according to claim 130, wherein the transcription-associated proteins are selected from RNA polymerase II, RNA polymerase II transcription factor B (TFIIB), RNA polymerase II transcription factor E, subunit β (TFIIEβ), acetylated histone H3 (AcH3), TATA-box binding protein (TBP), or CREB-binding protein (CBP).

135. The method according to claim 124, wherein the separating step (b) comprises physical separation of the complexes based on molecular size, charge, molecular weight, or recognition of molecular moieties.

136. The method according to claim 124, wherein the identifying step (c) comprises quantification of the number of nucleic acid sequences comprising the complexes.

137. The method according to claim 124, wherein identifying the nucleic acid components of the separated complexes comprises sequencing the nucleic acid molecules or a portion thereof; hybridizing the nucleic acid molecules to other known nucleic acid molecules, amplifying the nucleic acids, or generating a recombinant library from the isolated nucleic acid molecules, or portions thereof; and (i) sequencing the library or a portion thereof, or (ii) amplifying the nucleic acids, to determine if specific nucleic acid sequences are present in the isolated nucleic acid molecules.

138. The method according to claim 137, wherein generating a recombinant library comprises ligating random primers to the ends of the isolated nucleic acid molecules, or portions thereof; amplifying nucleic acid sequences corresponding to the isolated nucleic acid molecules or portions thereof; size-fractionating the amplified sequences to a desired size; concatamerizing the amplified molecules into chains of about 5-30 molecules; cloning the concatamerized molecules into a suitable cloning vector, growing the clones to obtain more copies thereof, and sequencing inserts of the clones.

139. The method according to claim 124, wherein identifying comprises amplifying isolated nucleic acids using primers specific for specific sequences in a genome.

140. The method according to claim 139, wherein amplifying involves PCR, quantitative PCR, ligation-mediated PCR, rolling circle amplification, transcription-mediated amplification, and ligase chain reaction.

141. The method according to claim 124, wherein identifying the protein components of the separated complexes comprises immunodetection, receptor-ligand binding, chemical methods, peptide sequencing, or array binding.

142. The method according to claim 141, wherein immunodetection is performed using antibodies directed toward one or more proteins of the complexes.

143. The method according to claim 141, wherein array binding is performed by (i) binding the proteins from the complexes onto an array comprising antibodies directed toward the protein in the complex, or (ii) binding nucleic acid from the complexes onto an array comprising nucleic acid molecules of known sequence.

144. The method according to claim 124, wherein the components of the complexes are stably associated before said isolating step (a).

145. The method according to claim 144, wherein cells are subjected to a cross-linking agent prior to said isolating step (a).

146. The method according to claim 144, wherein stable association of the components results from one or more of chemical cross-linking, biological cross-linking, ultraviolet light cross-linking, or cleavable linker interactions.

147. The method according to claim 146, wherein the cross-linking is reversible.
